(19) United States
(12) Patent Application Publication
(10) Pub. No.: US 2026/0232591 A1
(43) Pub. Date: Aug. 13, 2026

(54) BIODEGRADABLE MICROPARTICLES FOR SUSTAINED DRUG DELIVERY, METHODS OF PREPARATION AND USE THEREOF

(71) Applicant: OCULAR THERAPEUTIX, INC., Bedford, MA (US)

(72) Inventors: Peter JARRETT, Bedford, MA (US); Rami EL-HAYEK, Bedford, MA (US); Fadi HASO, Bedford, MA (US); Lokendrakumar BENGANI, Bedford, MA (US)

(21) Appl. No.: 19/156,565

(22) PCT Filed: Feb. 15, 2024
(86) PCT No.: PCT/US2024/015968

§ 371 Date: Aug. 14, 2025

Related U.S. Application Data
(60) Provisional application No. 63/446,419, filed on Feb. 17, 2023.

(51) Int. Cl. A61K 9/16; A61K 31/192; A61K 31/197; A61K 31/216; A61K 31/235; A61K 31/40; A61K 31/405; A61K 31/407; A61K 31/415; A61K 31/541; A61K 31/573; A61K 45/06

(52) U.S. Cl. CPC A61K 9/1647; A61K 9/1682; A61K 31/192; A61K 31/197; A61K 31/216; A61K 31/235; A61K 31/40; A61K 31/405; A61K 31/407; A61K 31/415; A61K 31/541; A61K 31/573; A61K 45/06

(57) ABSTRACT

In certain embodiments, the present invention relates to biodegradable microparticles for sustained release drug-delivery, comprising an active agent and a covalently and three dimensionally crosslinked polymer matrix, methods for preparing and use thereof. Furthermore, the present invention relates in certain embodiments to a pharmaceutically acceptable, sustained release, biodegradable drug-delivery’system, comprising biodegradable microparticles for sustained release drug-delivery, particularly for coating a medical implant or for use as a medical implant, and methods of manufacturing it. The present invention also relates in certain embodiments to corresponding methods of treatment, methods for controlling active agent release and uses of the biodegradable microparticles for sustained release drug-delivery.

### TECHNICAL FIELD

In certain embodiments, the present invention relates to biodegradable microparticles for sustained release drug-delivery, comprising an active agent and a covalently and three dimensionally crosslinked polymer matrix, methods for preparing and use thereof. Furthermore, the present invention relates in certain embodiments to a pharmaceutically acceptable, sustained release, biodegradable drug-delivery system, comprising biodegradable microparticles for sustained release drug-delivery, particularly for coating a medical implant or for use as a medical implant, and methods of manufacturing it. The present invention also relates in certain embodiments to corresponding methods of treatment, methods for controlling active agent release and uses of the biodegradable microparticles for sustained release drug-delivery.

### BACKGROUND

Controlled delivery of therapeutic agents is a large area of research in the recent years. A controlled delivery improves therapies, facilitates administration and leads to better compliance, less side effects and better therapeutic results.

Sustained delivery of hydrophilic drug compounds from a hydrogel-based implant or insert is often too fast or too slow for the desired treatment duration. This is because the rate of drug release from a water-based hydrogel increases as water solubility increases. It is desired to remove the solubility limitation.

Encapsulating drugs in microparticles of biodegradable polymers can be used to alter the release rate of the drugs included. For example, because the rate of drug release from a water-based hydrogel increases as water solubility increases, incorporation of drug encapsulating microparticles in such hydrogels allows for making the release of the drug more independent of the hydrogel degradation properties.

For delayed administration of active ingredients, a sustained release encapsulation of drugs, such as drug-loaded microparticles can be used to contain the drug compound and release it slowly. Commonly used encapsulants in forming microparticles are polymers of polylactic acid (PLA), polyglycolic acid (PGA) and polylactic-co-glycolic acid (PLGA). These materials are recognized as biodegradable and have been established as safe for use in humans and used in human clinical applications for several decades. There are numerous methods for creating such microparticles, typically involving precipitation of polymer microparticles from solutions of the polymer.

Microparticles for drug delivery have been described for example in US 2018/0085307, WO 2018/169950, WO 2021/237096, US 2021/0251893. For example, US 2018/0085307 and US 2021/0251893, which are incorporated herein by reference, describe treatments of ocular diseases with sustained release intracameral implants based on biodegradable hydrogels that include drug containing PLA microparticles. The PLA microparticles are prepared from drug containing solutions of polylactide polymer in an oil-in-water emulsion method.

For example, PLA or PLGA microparticles are prepared by rapidly solidifying PLA or PLGA from a solution of the polymer in an organic solvent in the presence of co-dissolved or microparticulate drug. The microparticles include the polymer in the form of physically aggregation of the polymer strands, and the polymers typically have acid or ester end groups. However, such conventionally prepared microparticles from solution-precipitated biodegradable polymers, copolymers or polymer blends, such as PLGA microparticles, are typically glassy materials once the residual solvent is removed. This property can make control of drug release difficult in vivo.

The glass transition temperature (Tg) is a characteristic property of the polymer, depending on composition and molecular weight. Also, Tg will decrease as degradation and moisture content increase after implantation. It has been observed that with these materials Tg often drops below body temperature, leading to a plastification of the microparticles, converting the glassy solid microparticle in vivo to a viscous liquid micro-droplet. This phase change during biodegradation has been found to be the cause of sometimes drastic changes in degradation kinetics and drug release rate in an uncontrolled manner, which is undesirable for a safe and reliable sustained release of the active agent.

For sustained release purposes, a zero-order release rate of the drug, i.e., a constant release rate that changes only slightly over time may be preferred in many cases. However, the release of drug from non-crosslinked, polymeric PLA or PLGA microparticles typically follows a sigmoidal curve that includes a lag phase, a release phase, and a decline phase, often showing a burst of drug directly after implantation. The release rates and the phases from PLA or PLGA microparticles depend on molecular weight, L to G ratio, the end group on the polymer (acid or ester) and environmental conditions. There is accordingly a need to provide microparticles for use in sustained drug delivery systems allowing to better control the release of active agents independent of their solubility in physiologic fluids, and producing reliable degradation kinetics of the polymer material used to encapsulate the active agent. There is also a need for providing microparticles for sustained release drug delivery that are heat stable and may be processed at high temperatures.

All references cited herein are incorporated by reference in their entireties for all purposes.

### OBJECTS AND SUMMARY OF INVENTION

It is thus an object of certain embodiments of the present invention to provide biodegradable microparticles for sustained release drug-delivery that can provide a substantially constant, zero-order release of the active agent over time.

It is another object of certain embodiments of the present invention to provide biodegradable microparticles for sustained release drug-delivery that show little or no burst of active agent release.

It is still another object of certain embodiments of the present invention to provide biodegradable microparticles for sustained release drug-delivery that are heat stable and can be processed at elevated temperatures, for example in hot melt extrusion processes.

It is an object of certain embodiments of the invention, and an aspect, to provide pharmaceutically acceptable, biodegradable microparticles for sustained release drug-delivery of an active ingredient to the body of a patient.

It is a further object of certain embodiments of the invention, and an aspect, to provide a method for manufacturing such biodegradable microparticles for sustained release drug-delivery of an active ingredient to the body of a patient.

It is a further object of certain embodiments of the invention, and an aspect, to provide a sustained release, biodegradable drug-delivery system, comprising the biodegradable microparticles for sustained release drug-delivery of the invention, particularly for use as a drug-eluting implant or for direct use as a medicament.

It is a further object of certain embodiments of the invention, and an aspect, to provide a method for controlling the release of an active agent from a sustained release, biodegradable drug-delivery system.

It is a further object of certain embodiments of the invention, and an aspect, to provide methods for treating a disease/medical condition of a patient using the biodegradable microparticles for sustained release drug-delivery of an active ingredient to the body of a patient.

It is a further object of certain embodiments of the invention, and an aspect, to provide a method for controlling the release of an active agent from a sustained release, biodegradable drug-delivery system comprising the biodegradable microparticles for sustained release drug-delivery of the present invention.

Some aspects of the present disclosure are directed to biodegradable microparticles for sustained release drug-delivery, comprising at least one active agent and a covalently and three-dimensionally crosslinked biodegradable polymer, wherein the crosslinked biodegradable polymer includes at least one of crosslinked polylactic acid (PLA), polyglycolic acid (PGA), polylactic-co-glycolic acid (PLGA), poly(vinylpyrrolidone), poly-p-dioxanone, poly(trimethylene carbonate), polycaprolactone, and/or polyvinyl alcohol, or copolymers of any of these.

Some aspects of the present disclosure are directed to biodegradable microparticles for sustained release drug-delivery, comprising at least one active agent and a covalently and three-dimensionally crosslinked biodegradable polymer, wherein the biodegradable microparticles include an organogel comprising at least one crosslinked polymer and an oil.

In some aspects of the present disclosure, the microparticles are microspheres having a substantially spherical shape.

In some aspects of the present disclosure, the active agent is dispersed, embedded or encapsulated in the organogel or polymer matrix forming the organogel. The organogel is formed by chemically crosslinking at least one multifunctional precursor to form the covalently and three dimensionally crosslinked biodegradable polymer matrix, optionally in the presence of an oil. In some aspects, the at least one multifunctional precursor has a functionality for chemical crosslinking of greater than 2, greater than 4, greater than 8, or from 2 to 16, 2 to 10 or 2 to 8. In some aspects, the at least one multifunctional precursor is a dendrimer or multi-arm precursor having a core and from 2 to 10 arms, or 3 to 10 arms, 4 to 8 arms, or 4 or 6 arms, each arm comprising a polymer unit and having a terminus. A functional group for chemical crosslinking may be bonded to each terminus.

In some aspects of the present disclosure the biodegradable, covalently crosslinked polymer of the microparticles comprises one or more polymer units of polyethylene glycol (PEG), polypropyleneglycol (PPG), polyvinyl alcohol, poly(vinylpyrrolidone), polylactic acid (PLA), polyglycolic acid (PGA), polylactic-co-glycolic acid (PLGA), poly-p-dioxanone, poly(trimethylene carbonate), polycaprolactone, and/or polyvinyl alcohol, or copolymers of any of these, random or block copolymers or combinations or mixtures of any of these, or one or more units of polyaminoacids, glycosaminoglycans, polysaccharides, or proteins, or combinations or mixtures of any of these. The biodegradable, covalently and three dimensionally crosslinked polymer matrix can comprise a plurality of hydrophobic polymer units and/or hydrophilic polymer units.

In some aspects of the present disclosure, the biodegradable, covalently and three dimensionally crosslinked polymeric comprises a plurality of hydrophobic polymer units such as polylactic acid (PLA), or polylactic-co-glycolic acid (PLGA) units, and/or hydrophilic polymer units such as polyethylene glycol units, polypropylene glycol units, or polyglycolic acid (PGA). In certain embodiments, the hydrophobic polymer units comprise polyethylene glycol units. In certain embodiments, the hydrophobic polymer units comprise polylactic acid (PLA) units.

In an embodiment of the present disclosure, the covalently and three-dimensionally crosslinked biodegradable polymer matrix comprises or consists of polylactic-co-glycolic acid (PLGA) units. The polylactic-co-glycolic acid (PLGA) units can have an L/G ratio (in % L or G units) ranging from about 0:100 to about 100:0, or about 1:99 to about 99:1, or about 10:90 to about 90:10, or about 25:75 to about 75:25. In certain embodiments, the L/G ratio is 50:50. In an aspect, each of the polymer units has an average molecular weight (Mw) in the range from about 1,000 to about 100,000 Daltons, or from about 10,000 to about 60,000 Daltons, or from about 15,000 to about 50,000 Daltons.

In some aspects of the present disclosure the polymer matrix is covalently crosslinked by hydrolysable bonds between polymer units intramolecular or intermolecular or a combination of both. For forming the organogel polymer matrix, at least one crosslinker having at least two functional groups, or more than two functional groups, such as a small molecule amine such as tris(2-aminoethyl)amine (TAEA) or trilysine may be used to crosslink the multifunctional precursor. Alternatively, or additionally, the organogel comprises or is formed of at least two crosslinkable dendrimer or multi-arm precursors intramolecularly crosslinked with each other. The dendrimer or multi-arm precursor can comprise a functional group on at least 3 of its arm termini, or on each terminus.

In some aspects of the present disclosure the polymer matrix is formed from at least two multi-arm precursors (e.g., 2 to 10 arm precursors), comprising a first multi-arm precursor comprising a first functional group, and a second multi-arm precursor comprising a second functional group, the functional groups being located at the terminus of the arms of the precursor or crosslinker, wherein the first or second functional group may be directly grafted to the precursor terminus, or via a linker molecule. In certain embodiments, each of the first functional group and the second functional group is selected from an electrophile and a nucleophile, functional groups for click chemistry, functional groups for cycloadditions, such as 1,3 dipolar cycloadditions, hetero-Diels-Alder cycloadditions, functional groups for nucleophilic ring openings, functional groups for non-aldol type carbonyl reactions, functional groups for addition reactions to carbon-carbon multiple bonds, polymerizable vinyl groups, or combinations thereof.

In an aspect of the present disclosure, each of the first functional group and the second functional group is selected from a group consisting of an electrophile and a nucleophile, and the reaction between the first functional group and second functional group is an electrophile-nucleophile reaction that forms a covalent bond, for example a polycondensation reaction. The nucleophile may be selected from one of an amine, such as a primary amine, a hydroxyl, an alcohol, a thiol, an azide anion, and a carboxyl group. The electrophile may be selected from activated ester groups such as succinimidyl esters, succinimidyl carbonates; nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halogen.

In an embodiment, and an aspect of the present disclosure, the nucleophile is an amine group, for example a primary amine, and the electrophile is an activated ester group, for example one of a succinimidyl ester selected from succinimidyl succinate, succinimidyl glutarate, succinimidyl adipate, succinimidyl azelate, or succinimidyl glutaramide.

In some other aspects of the present disclosure, each of the first functional group and the second functional group are selected from functional groups for click chemistry, including functional groups for cycloadditions, e.g., 1,3 dipolar cycloadditions, [3+2] cycloadditions such as alkene-nitrone cycloadditions or alkyne-nitrone cycloadditions, [4+2] cycloadditions, hetero-Diels-Alder cycloadditions; functional groups for thiol-ene reactions; functional groups for nucleophilic ring openings; functional groups for non-aldol type carbonyl reactions; functional groups for addition reactions to carbon-carbon multiple bonds; and functional groups for Michael-type additions.

In such aspects of the present disclosure, the first functional group is an alkyne compound such as a dibenzocyclooctyne (DBCO), or a bicyclo[6.1.0]-nonyne (BCN); or a norbornene, or a trans-cyclooctene (TCO), and the second functional group is an azide, a 3,4 dihydroxyphenylacetic acid (DHPA), or a tetrazine (Tz). The DBCO, BCN, norbornene, TCO, azide, DHPA and Tz functional groups may be grafted to the termini of the multi-arm precursor via a linker such as an acid group, a diacid group, a functionalized aliphatic, heteroaliphatic, or aromatic or heteroaromatic group.

In another aspect of the present disclosure, the first and second functional groups are selected for a [3+2] cycloaddition reaction such as alkene-nitrone cycloadditions or alkyne-nitrone cycloadditions.

In another aspect of the present disclosure, the first and second functional group are selected for a [4+2] cycloaddition reaction, particularly a hetero Diels-Alder reaction, wherein the first functional group is an aldehyde or imine compound, and the second functional group is a 1,3 diene compound, an unsaturated carbonyl compound, or a nitroso-alkene compound.

In a further aspect of the present disclosure, the first and second functional group are selected for a thiol-ene reaction, wherein the first functional group is a thiol compound and the second functional group is an alkene, such as a terminal alkene.

In other aspects of the present disclosure, the first and second functional group are selected for nucleophilic ring openings, wherein the first functional group is selected from an epoxide, thiirane, aziridine, or lactam, and the second functional group is nucleophile.

In an aspect of the present disclosure, the first and second functional group are selected for non-aldol type carbonyl reactions, wherein the first functional group is an aldehyde or ketone compound, and the second functional group is a primary amine, a hydrazide, acyl hydrazide or aminooxy compound, to form an imine, amide, isourea, hydrazone, acyl hydrazone or oxime linkage.

In another aspect of the present disclosure, each of the first functional group and the second functional group are selected from polymerizable vinyl groups and acrylates such as (meth)acrylic acid, (meth)acrylic acid esters, acrylamides, fumaric acid, maleic acid and combinations thereof.

In certain aspects of the present disclosure, crosslinking is induced thermally or photochemically with the use of electromagnetic radiation, optionally with the use of initiators such as photo initiators such as free radical photo initiators (Norish I type such as 2,2-dimethoxy-1,2-diphenyl-ethan-1-one, 2-Hydroxy-2-methyl-1-phenylpropanone, 1-hydroxy-cyclohexylphenylketone; or Norish II type such as benzophenone and its derivative and isopropyl thioxanthone in combination with a synergist such as tertiary amines2-ethylhexyl-(4-N,N-dimethyl amino)benzoate, and 2-ethyl-(4-N,N-dimethylamino)benzoate); or cationic photo-initiators.

In an aspect of the present disclosure, the organogel forming the biodegradable microparticles comprises a polymeric matrix, wherein the polymer is covalently crosslinked by linkages or bonds between polymer units. The linkages may be selected from amine, amide, urethane, ester, anhydride, ether, acetal, ketal, nitrile, isonitrile, isothiocyanate, isourea, hydrazone, oxime, or imine bonds, and combinations thereof.

In some aspects of the present disclosure the active agent is selected from at least one of a therapeutically active agent or a diagnostically active agent, or combinations thereof. The therapeutically active agent may be selected from steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, viruses, viruses for gene delivery such as AAV, etc., or any combinations thereof.

In certain aspects of the present disclosure, the microparticles have a particle size (diameter) of about 0.1 μm to about 1000 μm, or about 1 μm to about 150 μm, about 1 μm to about 100 μm, about 20 μm to about 75 μm, about 10 μm to about 106 μm or about 20 μm to about 55 μm, determined by sieving, or have an average diameter ranging from about 0.1 μm to about 1000 μm, or about 1 μm to about 150 μm, about 1 μm to about 100 μm, about 20 μm to about 75 μm, about 10 μm to about 106 μm or about 20 μm to about 55 μm, determined by laser diffraction, and assuming substantially spherical shape. The microparticles can have a particle size distribution such as a D50 particle size of less than about 100 μm, or less than about 50 μm, or less than about 20 μm and/or a D90 particle size of less than about 200 μm, or less than about 50 μm, or a D90 particle size of about 100 μm or less, or 30 μm or less and/or a D90 particle size of about 20 μm or less, determined by laser diffraction. In certain embodiments, the lower limit of D50 and D90 is 1 μm, 5 μm or 10 μm and can be a range with any of the values above.

In some aspects of the present disclosure, the biodegradable microparticles are comprising or are consisting of a blend of microparticles having different particle sizes and/or having a different polymer matrix, and/or including different active agents.

In some aspects of the present disclosure, the selection of the organogel precursors, and/or the hydrophobicity of the polymer units, and/or the L/G ratio of PLGA units is used to tune the release rate. For example, combinations of PEG and PLA or PLGA precursors in varying amounts can be used to adjust the lipophilicity of the microparticles.

In an aspect of the present disclosure, the biodegradable microparticles provide for a release of an effective amount of the active agent for a period of time, such as up to about 1 year, up to about 9 months, up to about 6 months, up to about 3 months, up to about 1 month, or up to about 25 days after administration, such as up to about 14 days, or up to about 21 days after administration, wherein optionally the active agent release is substantially constant in a temperature range of about 30° C. to about 45° C., or about 36 to about 43° C.

In some aspects of the present disclosure the polymer matrix has a glass transition temperature below human body temperature, such as below about 37° C., or below about 36° C., below about 30° C., below about 25° C., below about 20° C., or below about 10° C., and/or wherein the polymer matrix has a melt temperature above about 40° C., about 45° C., about 50° C., about 60° C. or about 70° C. In certain embodiments, the lower limit of glass transition temperature is about 5° C. or about 10° C. or about 20° C. or about 30° C. and can be a range with any of the above values. In certain embodiments, the polymer matrix has a melt temperature no more than about 50° C. or about 75° C. or about 100° C. or about 150° C. and can be a range with any of the above values.

Some aspects of the present disclosure are directed to a method for manufacturing the biodegradable microparticles for sustained release drug-delivery as disclosed herein, the method being selected from, e.g., one of the following methods: emulsion solvent evaporation-extraction, emulsion solvent diffusion, supercritical fluid emulsion, coacervation, spray drying, hydrogel template, microfluidic systems, membrane extrusion emulsification, particle replication in non-wetting templates (PRINT) technique, electro hydrodynamic atomization (EHDA) or electro-spraying, or particles obtained from gas saturated solutions (PGSS) method, or by 3D printing.

In some aspects of the present disclosure are directed to a method for manufacturing the biodegradable microparticles for sustained release drug-delivery as described herein, the method comprising the steps of: (1) forming a gel comprising a covalently crosslinked polymer in the presence of at least one active agent, optionally at least one oil and optionally a first solvent, (2) producing microparticles wherein the at least one active agent is dispersed within the covalently crosslinked polymer, and (3) optionally removing solvent.

In some aspects of the present disclosure, the above method comprises the steps of (a) dissolving at least one of the polymer precursors in a first solvent, producing a first mixture; (b) b) providing a second mixture comprising a crosslinker in a second solvent; (c) adding at least one active agent, and optionally an oil, to at least one of the first mixture or second mixture; (d) combining the first mixture and the second mixture to produce a first phase; (e) providing a second phase comprising a third solvent, that is immiscible with the first and second solvents; (f) introducing, under agitation, the first phase into the second phase, thereby producing an emulsion of dispersed first phase in the second phase; and (g) removing the first, second and/or third solvent. These steps can be performed in any order.

The step of producing microparticles (step (2)) or the step (f) comprises forcing the first phase through a mesh or injecting the first phase into the agitated second phase, the first and/or second solvent and/or third solvent optionally comprising additives such as emulsifiers, surfactants, dispersing adjuvants, or porogens, to form microspheric or nanospheric particles.

In some aspects of the method of the present disclosure, the first or second solvent is selected from acetone, acetonitrile, benzyl alcohol, chloroform, dichloromethane (DCM), dioxane, dimethyl carbonate, DMSO, ethanol, ethyl acetate, ethyl formate, ethyl propionate, glycofurol, hexafluoro-isopropanol, isosorbide dimethyl ether, isopropanol, methyl chloride, methylene chloride, methyl ethyl ketone, N-methylpyrrolidone, propylene carbonate, or tetrahydrofuran, or any mixtures thereof, and the third solvent is water, an alcohol such as methanol, ethanol or propanol, or any mixture thereof.

In some aspects of the method of the present disclosure, additives may be used, such as additives selected from surfactants or emulsifiers such as polyvinyl alcohol (PVA), polyethylene glycol sorbitan monolaurate (Tween®), sorbitan monolaurate (Span®), sodium dodecyl sulfate (SDS); and/or porogens such as inorganic salts (NaCl, KCl, sodium or potassium carbonates or bicarbonates, ammonium bicarbonate), Pluronics; sodium or potassium oleate; gelatin; mustard oil, mineral oil; cyclodextrins; carbohydrates, bovine serum albumin (BSA); photo initiators, radical polymerization initiators, and combinations thereof.

In some aspects of the method of the present disclosure, steps (1) and (2) make use of oil-in-water emulsion or water-in-oil emulsion technology, or combinations thereof, such as single or double emulsion technique, or microfluidic technology, or combinations thereof.

In some aspects of the method of the present disclosure, removal of the first, and/or second and/or third solvent is done by one of hot air convection or direct drying, indirect or contact drying, spray drying, dielectric drying, vacuum drying, freeze drying, supercritical or superheated steam drying, or combinations of any of these.

Some aspects of the present disclosure are directed to a sustained release, biodegradable drug-delivery system comprising the biodegradable microparticles for sustained release drug-delivery as disclosed herein. In some aspects, the biodegradable microparticles are incorporated into a hydrogel, xerogel or organogel, optionally by using extrusion methods, such as extrusion or injection molding of a reaction mixture comprising the biodegradable microparticles of the present disclosure dispersed in a hydrogel, xerogel or organogel or precursors thereof for in situ implant forming. In some aspects thereof, the gelling occurs before and/or during extrusion or injection molding of the gel-forming mass comprising the biodegradable microparticles.

In other aspects, the sustained release, biodegradable drug-delivery system of the present disclosure is used for coating a medical implant or for use as a medical implant. The implant may be selected from intraocular implant, intracaveal implant, intracameral implant, an implant for introduction into the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrascleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroid, tenon, cornea, cancer tissue, organ, prostate, breast, joint space, subdural, dental, subcutaneous, carpal tunnel, perivascular, surgically created space or injury, void space, and potential space.

Some aspects of the present disclosure are directed to a sustained release, biodegradable drug-delivery system comprising the biodegradable microparticles as disclosed herein or manufactured by a method as disclosed herein, for use as a medicament.

In some aspects the present invention is directed to a sustained release, biodegradable drug-delivery system comprising the biodegradable microparticles as disclosed herein or manufactured by a method as disclosed herein, for use in treating a disease/medical condition of a patient, the use comprising the incorporation of biodegradable microparticles of the present disclosure into a carrier such as a hydrogel, organogel or xerogel, wherein the hydrogel, organogel or xerogel is formed in situ at a treatment site of the patient, or is prefabricated and delivered to or implanted at a treatment site of the patient in order to release the active agent from the microparticles over an extended period of time, or the carrier being a solvent or solvent system to produce an injectable suspension or dispersion.

In some aspects the present invention is directed to a sustained release, biodegradable drug-delivery system comprising the biodegradable microparticles as disclosed herein or manufactured by a method as disclosed herein, method for treating a disease/medical condition of a patient, the method comprising the incorporation of biodegradable microparticles according to the present disclosure into a hydrogel, organogel or xerogel, wherein the hydrogel, organogel or xerogel is formed in situ at a treatment site of the patient, or is prefabricated and delivered to or implanted at a treatment site in order to release the active agent over an extended period of time.

In some aspects the present invention is directed to a method for treating a disease/medical condition of a patient, the method comprising administering a hydrogel, organogel or xerogel comprising the biodegradable microparticles according to the present disclosure to the patient in order to release the active agent over an extended period of time.

In some aspects the present invention, the treatment site is selected from the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrascleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroidal, tenon, cornea, cancer tissue, organ, prostate, breast, joint, subdural, dental, subcutaneous, carpal tunnel, perivascular, surgically created space or injury, void space, and potential space.

In some aspects, the disease/medical condition to be treated is an eye disease, such as back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy; or glaucoma, ocular hypertension, hyphemia, presbyopia, cataract, retinal vein occlusion, inflammation.

In some aspects, the present disclosure is further directed to a method for controlling the release of an active agent from a sustained release, biodegradable drug-delivery system as disclosed herein or manufactured in by a method as disclosed herein, the control of active agent release comprising either one or a combination of the following measures: Selecting the L/G ratio of the polylactic-co-glycolic acid (PLGA) units to adjust the hydrophobicity of the polymer matrix forming the microparticles; selecting the L/G ratio of the polylactic-co-glycolic acid (PLGA) units to provide a sustained release of the active agent from the microparticles; selecting the molar ratio of the amounts of the first to second crosslinkable precursors to adjust the hydrophobicity of the polymer matrix forming the microparticles; selecting the molar ratio of the amounts of the first to second crosslinkable precursors to provide a sustained release of the active agent from the microparticles; selecting the amount and/or particle size of the biodegradable microparticles to be included in the in the hydrogel, organogel xerogel; adding a third crosslinkable precursor that is less hydrolysable than the first and second, optionally varying the molar ratios of the first, second and/or third precursors upon forming the biodegradable microparticles; dispersing an active agent that has high water solubility in particulate form into the organogel of a biodegradable microparticles.

### Definitions

The term “biodegradable” refers to a material or object (such as the microparticles according to the present invention) which becomes degraded in vivo, i.e., when placed in the human or animal body or in vitro when immersed in an aqueous solution under physiological conditions such as pH 7.2-7.4 at 37° C. In the context of the present invention, as disclosed in detail herein below, the microparticles within which an active agent is contained, slowly biodegrade over time once administered or deposited in the human or animal body. In certain embodiments, biodegradation takes place at least in part via ester hydrolysis in the aqueous environment of the body. Biodegradation may take place by hydrolysis or enzymatic cleavage of the covalent crosslinks and/or within the polymer units. The microparticles slowly soften and disintegrate, resulting in clearance through physiological pathways. In certain embodiments, the microparticles of the present invention retain their shape over extended periods of time (e.g., about 1 month, 3 months or 6 months). In certain embodiments, the shape is maintained due to covalent crosslinking of the polymer components forming the microparticles, e.g., until the active agent or at least a major amount (e.g., at least 50%, at least 75% or at least 90%) thereof has been released therefrom.

In embodiments of this invention, the microparticles include an organogel. An “organogel” in the present invention is a solid or semi-solid system forming a covalently crosslinked three-dimensional network of one or more hydrophilic or hydrophobic natural or synthetic polymers (as disclosed herein) that include an oil or generally a hydrophobic organic liquid as disclosed herein. Thus, in the present invention “organogels” are limited to so-called chemical organogels, wherein the intermolecular interaction between organogelator molecules is a chemical linkage (e.g., covalent bond) that is formed during gelation by chemical reactions inducing crosslinking. The “organogel” as used herein refers to a three-dimensional polymer network or matrix of at least two precursors/gelators/precursors that are covalently cross-linked with each other in the presence of an oil and optionally an organic solvent and comprising the oil contained within the covalently crosslinked polymer of which the microparticles are formed of.

A “hydrogel” is a three-dimensional network of one or more hydrophilic natural or synthetic polymers (as disclosed herein) that can swell in water and hold an amount of water while maintaining or substantially maintaining its structure, e.g., due to chemical or physical cross-linking of individual polymer chains. Due to their high-water content, hydrogels are soft and flexible, which make them similar to natural tissue. In the present invention the term “hydrogel” is used to refer both to a hydrogel in the hydrated state when it contains water (e. g. after the hydrogel has been formed in an aqueous solution, or after the hydrogel has been hydrated or (re-)hydrated once inserted into the body or otherwise immersed into an aqueous environment) and to a hydrogel in its dry (dried/dehydrated) state, e.g., when it has been dried to a low water content of e.g. not more than 1% by weight or when the preparation results in a low water content insert without the necessity of a drying step.

The terms “polymer”, “polymer network” or “polymer matrix” used in the context of the microparticles of the invention describes a structure formed of polymer chains (of the same or different molecular structure and of the same or different molecular weight) that are covalently cross-linked with each other. The types of polymers suitable for the purposes of the present invention are disclosed herein below. The term “polymer network” is used interchangeably with the term “matrix”.

The term “amorphous” refers to a polymer or polymer network, which does not exhibit a melting point or crystalline structures in X-ray or electron scattering experiments.

The term “semi-crystalline” refers to a polymer or polymer network, which possesses some crystalline character, i.e., exhibits a melting point or some crystalline properties in X-ray or electron scattering experiments.

The term “precursor” or “gelator” or “component” herein refers to those molecules or compounds that are reacted with each other and that are thus connected via covalent crosslinks to form a polymer network, and optionally an organogel matrix when oil is present, forming the microparticles.

The parts of the precursor molecules that are still present in a final polymer are also called “units” herein. The “units” are thus the building blocks or constituents of a polymer network forming the microparticles. For example, a polymer network suitable for use in the present invention may contain identical or different PLGA units, polyethylene glycol units, or other types of polymers as further disclosed herein.

The term “release” (and accordingly the terms “released”, “releasing” etc.) as used herein refers to the provision of active agents from the microparticles or a drug-delivery system such as an implant including the microparticles of the present invention to the surrounding environment. The surrounding environment may be an in vitro or in vivo environment as described herein. In certain specific embodiments, the surrounding environment is the vitreous humor and/or ocular tissue, such as the retina and the choroid.

The term “100% release of the active agent” should be construed as from 95% to 100%. The way this controlled release is achieved is by a number of parameters that are characteristics of the drug-delivery system as disclosed herein. Each such characteristic feature of the drug-delivery system alone or in combination with each other can be responsible for the controlled release.

The term “sustained release” for the purposes of the present invention is meant to characterize products such as the biodegradable microparticles, which are formulated to make an active agent available over an extended period of time, thereby allowing a reduction in dosing frequency compared to an immediate release dosage form, such as for example a solution of an active agent that is topically applied onto the eye (i.e. eye drops). Other terms that may be used herein interchangeably with “sustained release” are “extended release” or “controlled release”. Within the meaning of the invention, the term “sustained release” comprises constant active agent release, tapered active agent release, ascending active agent release as well as any combination thereof such as a constant active agent release followed by a tapered active agent release. Within the meaning of the invention, the term “tapered”, or “tapering” refers to a decrease of active agent release over time. Specifically, the term “sustained release” refers to release of an active agent from the microparticles or drug-delivery system including them in a predetermined way and is in contrast to an immediate release like a bolus injection. In certain embodiments, the controlled release refers to the amount of the active agent release over the total number of days required for 100% release of the active agent in an aqueous solution under in-vitro physiological conditions such as at pH 7.2-7.4 and 37° C.

The term “extended period of time” as used herein refers to any period of time that would be considered by those of ordinary skill in the art as being extended with respect to treating a disease, and in particular refers to periods such as at least about 1 week, or at least about 1 month or longer, such as up to about 12 months, or any intermediate periods such as about 1 to about 6 months, about 2 to about 4 months, about 2 to about 3 months or about 3 to about 4 months or as otherwise disclosed herein.

A “zero order” release or “substantially zero order” release or “near zero order” release is defined as exhibiting a relatively straight line in a graphical representation of percent of the active agent released versus time. In certain embodiments of the present invention, substantially zero order release is defined as the amount of the active agent released which is proportional within 20% to elapsed time.

The terms “API”, “active (pharmaceutical) ingredient”, “active (pharmaceutical) agent”, “active (pharmaceutical) principle”, “(active) therapeutic agent”, “active”, and “drug” are used interchangeably herein and refer to the substance used in a finished pharmaceutical product (FPP) as well as the substance used in the preparation of such a finished pharmaceutical product, intended to furnish pharmacological activity or to otherwise have direct effect in the diagnosis, cure, mitigation, treatment or prevention of a disease, or to have direct effect in restoring, correcting or modifying physiological functions in a patient.

The active agent used according to the present invention may be an active agent for the treatment and/or prevention of a disease or disorder, or a diagnostic agent such as a marker. In an embodiment of the invention, the active agent is a low water solubility active agent (i.e., having a solubility in water of less than about 1000 g/mL or less than about 100 μg/mL). In other embodiments of the invention, the active agent is a highly water-soluble active agent (i.e., having a solubility in water of greater than about 1000 μg/mL or even greater than 10 mg/mL). This definition is not dependent on the agent being approved by a governmental agency.

For the purposes of the present invention, an active agent in all its possible forms, including free acid, free base, polymorphs or any pharmaceutically acceptable salts, anhydrates, hydrates, co-crystals, or other solvates or derivatives, such as pro-drugs or conjugates, can be used. Whenever in this description or in the claims an active agent is referred to without further specification, even if not explicitly stated, it also refers to the active agent in the form of any such polymorphs, pharmaceutically acceptable salts, anhydrates, or solvates (including hydrates) (let's discuss this deletion) thereof. With respect to the active agent, suitable solid forms include without limitation the pure substance form in any physical form known to the person of ordinary skill in the art. For example, the active agent may be in the form of particles. Particles can be amorphous or crystalline, or present a mixture of the two forms, and can be made of any size which could be without limitation classified as coarse, fine or ultrafine particles, the dimensions of which may be in particular visible to the naked eye or under the microscope and have shapes such as single grains and/or agglomerates. Particles may also be micronized. As used herein, the term “micronized” refers to small-size particles, in particular those of microscopic scale, which are without limitation reduced in particle size, by e.g., jet milling, jaw crushing, hammer milling, wet milling, precipitation in non-solvent, cryomilling (milling with liquid nitrogen or dry ice) and ball milling. An active agent can also be present in dissolved or dispersed state, e.g., within a solvent or in an aqueous medium, for example in the form of particles dispersed in an oil, or a compatible aqueous suspension which may optionally include further excipients such as a surfactant.

As used herein, the term “therapeutically effective” refers to the amount of active agent needed to produce a desired therapeutic result after administration. For example, in the context of the present invention, one desired therapeutic result would be the reduction of symptoms associated with DED, e.g., as measured by in vivo tests known to the person of ordinary skill in the art, such as an increase of a Schirmer's tear test score, a reduction of Staining values as measured by conjunctival lissamine green staining or corneal fluorescein staining, a reduction of the eye dryness severity and/or eye dryness frequency score on a visual analogue scale (VAS), a reduction of the Ocular Surface Disease Index and/or the Standard Patient Evaluation of Eye Dryness score as well as a reduction of the best corrected visual acuity. In one embodiment, “therapeutically effective” refers to an amount of active agent in a sustained release intracanalicular insert capable of achieving a tear fluid concentration which is equivalent in terms of therapeutic effect to a cyclosporine concentration of 0.236 μg/mL (which is considered to be required for immunomodulation, Tang-Liu and Acheampong, Clin. Pharmacokinet. 44 (3), pp. 247-261)) over an extended period of time and in particular over substantially the whole remaining wearing period of the insert once said tear fluid concentration is achieved.

As used herein, the values “d10”, “d50”, “d90” and “d100” refer to a value characterizing the proportion of particles in a particle size distribution meeting a certain particle size. In a given particle size distribution, 10% of the particles present a particles size of d10 or less, 50% of the particles present a particles size of d50 or less, 90% of the particles present a particles size of d90 or less, and substantially all particles present a particles size of d100 or less. The percentages may be given by different parameters known to the person of ordinary skill in the art, e.g., the percentages may be based on volume, weight, or the number of the particles. Thus, d50 may exemplarily be the volume-based, the weight-based or the number-based median particle size. For example, a volume-based d90 of 43 μm means that 90% of the particles by volume have a particle size of 43 μm or less. In certain embodiments, the d10, d50 and d90 are volume-based values. The particle size distribution PSD can be commonly measured by methods as known to the person of ordinary skill in the art and includes sieving as well as laser diffraction methods. In certain embodiments, the PSD is measured by laser diffraction in accordance with USP <429> Light Diffraction Measurement of Particle Size. In certain embodiments, the PSD is measured by laser diffraction using a Beckman Coulter LS 13 320 based on the optical model “Fraunhofer.rf780z” with an obscuration value ranging from 7 to 9%.

The term “patient” herein includes both human and animal patients. The biodegradable drug-delivery systems according to the present invention are therefore suitable for human or veterinary medicinal applications. Generally, a “subject” is a (human or animal) individual to which a drug-delivery systems according to the present invention is administered. A “patient” is a subject in need of treatment due to a particular physiological or pathological condition. A “patient” does not necessarily have a diagnosis of the particular physiological or pathological condition prior to receiving the drug-delivery system.

The molecular weight of a polymer precursor as used for the purposes of the present invention and as disclosed herein may be determined by analytical methods known in the art. The molecular weight of polyethylene glycol may for example be determined by any method known in the art, including gel electrophoresis such as SDS-PAGE (sodium dodecyl sulphate-polyacrylamide gel electrophoresis), gel permeation chromatography (GPC), including GPC with static light scattering detectors (SLS) or dynamic light scattering (DLS), liquid chromatography (LC), as well as mass spectrometry such as matrix-assisted laser desorption/ionization-time of flight (MALDI-TOF) spectrometry or electrospray ionization (ESI) mass spectrometry. The molecular weight of a polymer, including a polyethylene glycol precursor as disclosed herein, is an average molecular weight (based on the polymer's molecular weight distribution), and may therefore be indicated by means of various average values, including the weight average molecular weight (Mw) and the number average molecular weight (Mn). In the case of the crosslinkable polymer gelators, such as polyethylene glycol, PLGA and poloxamer-based precursors as used in the present invention, the molecular weight indicated herein is the number average molecular weight (Mn) determined by gel permeation chromatography using a polystyrene standard, according to standard methods known in the art. Typically, the materials, especially the multi-arm precursors are purchased with a specified molecular weight defined by the vendor. Suitable PEG precursors are for example available from a number of suppliers, such as Jenkem Technology and others.

The term “day 1” as used herein refers to a time point that immediately follows after “day 0”. Thus, whenever “day 1” is used, it refers to an already elapsed time period of one day or about 24 hours after administration of the drug-delivery system.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that.

The term “average” as used herein refers to a central or typical value in a set of data (points), which is calculated by dividing the sum of the data (points) in the set by their number (i.e., the mean value of a set of data).

As used herein, the singular forms “a,” “an,” and “the” include plural references unless the context clearly indicates otherwise.

The term “and/or” as used in a phrase such as “A and/or B” herein is intended to include both “A and B” and “A or B”.

Open terms such as “include,” “including,” “contain,” “containing” and the like mean “comprising.” These open-ended transitional phrases are used to introduce an open-ended list of elements, method steps, or the like that does not exclude additional, unrecited elements or method steps.

The term “up to” when used herein together with a certain value or number is meant to include the respective value or number.

The terms “from A to B”, “of from A to B”, and “of A to B” are used interchangeably herein and all refer to a range from A to B, including the upper and lower limits A and B.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

The abbreviation “PBS” when used herein means phosphate-buffered saline.

The abbreviation “PEG” when used herein means polyethylene glycol.

The abbreviation “PLGA” when used herein means poly(lactic-co-glycolic acid). If not specified otherwise, it has a L/G ratio of 1:1 (50:50)

The term “hydrophobic”, or “lipophilic” is defined as a property of polymers or materials having a low degree of water attraction or absorption, i.e. the material is repelled from a mass of water. The term “hydrophilic”, or “lipophobic” is vice versa defined as a property of materials or polymers that attract water or have a strong affinity for water. Hydrophobicity may be measured by determining contact angles of drops of liquids, preferably water droplets, formed on a solid polymer and/or gel surface. Furthermore, hydrophobic organic liquids as used in the present invention are immiscible or at least not readily miscible with water.

A “hydrophilic” molecule, e.g., a precursor or precursor portion, has a solubility of at least 1 g/100 mL in an aqueous solution.

The term “immobilized” as used herein refers to long range immobility and does not refer to localized mobility within the microparticle polymer matrix, for example, the oil is present as a continuous phase within the polymer matrix and may be slowly mobile only in vivo, i.e., it can slowly escape into body fluid over time.

### BRIEF DESCRIPTION OF THE DRAWINGS

FIG. 1 illustrates an example for optimizing drug release by using a blend of different prior art non-crosslinked polymer microparticles versus the release characteristics of the individual prior art non-crosslinked microparticles made from different types of PLA polymers.

FIG. 2 is an image of the used Fibrijet® Y type mixer.

FIG. 3 is a SEM image of crosslinked PLGA microparticles without an active agent after heating at 80° C. for 2 hours.

FIG. 4 shows SEM images of microparticles of embodiments of the invention with a diameter>106 μm before and after heating at 80° C. for 2 hours.

FIG. 5 shows SEM images of microparticles of embodiments of the invention with a diameter between 20-106 μm before and after heating at 80° C. for 2 hours.

FIG. 6 shows microscope images of the microparticles of embodiments of the invention (left) and comparative, non-crosslinked PLA microparticles (right) after the heat treatment at 80° C. for 2 h.

FIG. 7 illustrates the influence of diameter and heat treatment of microparticles on the in-vitro release of travoprost.

FIG. 8 is a graph of the in-vitro release of travoprost from crosslinked PLGA microparticles compared to prior art non-crosslinked PLA-microparticles.

FIG. 9 is a graph of the in-vitro release of travoprost of comparative, non-crosslinked PLA microparticles at different temperatures.

### DETAILED DESCRIPTION OF THE INVENTION

The present invention is directed to pharmaceutically acceptable, biodegradable microparticles for sustained release drug-delivery of an active ingredient to the body of a patient. In a further aspect, a method for manufacturing such biodegradable microparticles for sustained release drug-delivery of an active ingredient to the body of a patient are provided. In certain aspects, the biodegradable microparticles can be incorporated into sustained release, biodegradable drug-delivery systems, particularly for use as a drug-eluting implant, or the microparticles are for direct use as a medicament, such as injection solutions comprising the biodegradable, drug-eluting microparticles in suspended form.

In an embodiment, the biodegradable microparticles for sustained release drug-delivery comprise at least one active agent, such as a drug and a covalently and three-dimensionally crosslinked biodegradable polymer. The active agent can be, e.g., dispersed, embedded or encapsulated in the covalently crosslinked biodegradable polymer.

### Microparticles

The biodegradable microparticles in certain embodiments are formed by covalently crosslinking multifunctional monomeric, oligomeric or polymeric precursor molecules as disclosed herein later via formation of chemical bonds or linkages. A three dimensionally covalently crosslinked polymer network may be formed that includes the active agent (and other optional ingredients) and immobilizes it within the polymeric network of the microparticles, e.g., until it is released from the microparticles in vivo or in vitro. A solvent or a hydrophobic organic liquid, such as an oil, can additionally be present in the microparticles, resulting in an organogel microparticle.

Covalent crosslinking of the polymer forming precursors in certain embodiments provides a limited mobility to active agent dispersed or encapsulated therein. This provides continuous control of drug release by limiting drug transport mainly to diffusion through the polymer matrix of the microparticle, that may be largely independent of the degradation rate of the polymer itself. Furthermore, the development of defects in the polymer, for example by plastization, that provide fast escape routes for the drug are eliminated from developing in such crosslinked polymer microparticles. In certain embodiments, the biodegradable microparticles of the present invention are a fully or at least partly diffusion controlled delivery system, i.e. the release of the active agent from the microparticles is primarily controlled by diffusion processes. In certain embodiments, in vivo degradation of the polymer additionally occurs in the microparticles of the present invention, but does not primarily control the release of the active agent. In non-crosslinked microparticles from solvent precipitation of linear polymers the release of the active agent is mainly controlled by degradation of the polymer matrix that releases the active agent mainly in a degradation controlled system.

In certain embodiments, the use of a covalently crosslinked polymer in the biodegradable microparticles for drug-delivery of the invention thus allows to modify the release of an active agent from the microparticles or a drug-delivery system including the microparticles by tailoring or suitably selecting the precursor components forming the crosslinked polymer according to their hydrophilic and/or hydrophobic properties.

Furthermore, in certain embodiments, the release of an active agent from the microparticle or drug-delivery system can be modified or controlled by suitably selecting types and amounts of additives such as oils (hydrophobic organic liquids), for example selected according to their properties such as hydrophobicity, viscosity, compatibility with the active agent, solubility or insolubility of the active agent in the oil, and the like.

In embodiments where oils are used in the biodegradable microparticles, the crosslinked polymer forms an organogel including the oil in the crosslinked polymer matrix. The oil may include the active agent in dissolved or dissolved form, and can be used to modify the release of the active agent or to remove incompatibilities of the active agent with the polymer. In other such embodiments, the active agent may be an oil itself, resulting in organogels as the biodegradable microparticles. The hydrophobic organic liquid can also act as a drug cosolvent during production of the microparticles.

The biodegradable microparticles of certain embodiments of the present invention offer several advantages over direct incorporation of active agents into hydrogels. For example, microparticles can be made from hydrophobic polymers and may be anhydrous, so water degradable (hydrolysable) components such as water sensitive active agents can be stabilized and made storage stable by encapsulation in biodegradable microparticles over extended periods of time and do not require hydration at the time of implantation.

Water soluble compounds have low solubility or are insoluble in more hydrophobic polymers, allowing the drug to be incorporated as a particulate solid embedded in the microparticle for leaching out by body fluids in vivo. The low solubility of a drug in the microparticle polymer thus provides a reliable mechanism to control the rate of drug release. This property vastly increases the range of compounds that can be contained in an implant.

Further, manipulation of the lipophilicity/hydrophilicity of the polymer of the microparticles can be used to adjust the release rate of a drug and to influence diffusion rates. Simple hydrogels cannot be adjusted this way, since they are water-based, thus in these systems the drug itself has to be modified to a prodrug form for such adjustment of drug/matrix solubility. The use of active agent loaded microparticles as described herein, embedded in a hydrogel matrix of an implant, solubility problems and the use od prodrugs can be avoided. Additionally, varying the lipophilicity/hydrophilicity of the polymer can be further used to influence the degradation rate of the polymer matrix itself, also having an additional influence on the release rate of a drug from the microparticle.

During preparation of the microparticles, solvent can be involved that has to be removed afterwards. Removal of the solvent can be accomplished, e.g., by heat treatment, lyophilization, evaporation, or vacuum drying. Some of these drying treatments are limited or even not possible for non-crosslinked materials that undergo melting or glass transitions at elevated temperatures. Others, such as lyophilization are costly. In certain embodiments of the present invention, covalently crosslinked biodegradable polymers are used, yielding heat-stable biodegradable microparticles.

In certain embodiments, microparticles can be dimensionally stable to heat and will not melt, e.g. up to temperatures of about 50° C., 60° C., 70° C., 80° C., 90° C. or about 100° C. Solvent extraction methods requiring heat treatments can be used without affecting the release properties of the microparticles. Additionally, their heat stability does allow the microparticles to be used in extrusion processes, such as hot melt extrusion, or in 3D printing, for example during inclusion in hydrogels or organogels for producing composite implants.

In certain embodiments, during microparticle production, the particles harden or cure by crosslinking, not only by solvent removal in emulsion processes such as used with non-crosslinking polymers. This makes the process of microsphere production faster and simpler.

In certain embodiments, the microparticles further possess the physical qualities of low modulus, dimensional stability, and favorable drug release kinetics. It has also been observed that the microparticles of certain embodiments of the embodiments of the invention reveal release profiles that are substantially independent of temperature, i.e. not affected by temperature changes as they occur under physiological conditions.

The microparticles can have regular or irregular shape. In one general embodiment of the present disclosure, the microparticles are microspheres, the microspheres having a substantially spherical shape as it is typically obtained by the microparticle manufacturing methods as described herein.

In one embodiment, the microparticles have a particle size (diameter) of about 0.1 μm to about 1000 μm, or about 1 μm to about 150 μm, about 1 μm to about 100 μm, about 20 μm to about 75 μm, about 10 μm to about 106 μm or about 20 μm to about 55 μm, determined by sieving, or have an average diameter ranging from about 0.1 μm to about 1000 μm, or about 1 μm to about 150 μm, about 1 μm to about 100 μm, about 20 μm to about 75 μm, about 10 μm to about 106 μm or about 20 μm to about 55 μm, determined by laser diffraction.

In one embodiment, the biodegradable microparticles have a particle size distribution such as a D50 particle size of less than about about 100 μm, or less than about about 50 μm, or less than about about 20 μm and/or a D90 particle size of less than about about 200 μm, or less than about about 50 μm, or a D90 particle size of about about 100 μm or less, or about 30 μm or less and/or a D90 particle size of about about 20 μm or less, determined by laser diffraction. Can we add optional lower limits?

In certain embodiments, the biodegradable microparticles consist or consist essentially of at least one active agent and at least one covalently crosslinked biodegradable polymer. In other embodiments, the biodegradable microparticles comprise at least one active agent and the covalently crosslinked biodegradable polymer, and further additives may be present in the biodegradable polymer.

Further additives for use in the biodegradable microparticles of embodiments of the invention include hydrophobic organic liquids such as an oil, solvents, salts, porogens, buffers, non-crosslinked oligomers or polymers, sugars, visualization agents, markers, etc.

The microparticles can be formed by crosslinking a biodegradable polymer precursor, such as functionalized PLA, PLGA or other polymers as disclosed herein, e.g., in the presence of co-dissolved or microparticulate drug and a solvent. These microparticles can be composed of the crosslinked polymer precursor alone, or of an oil/polymer blend. In certain embodiments, gelation into an organogel eliminates the need for rapid solvent removal and results in a rubbery material, i.e. a material that is above its glass transition temperature. PLGA microparticles are typically glassy materials at room temperature once any residual solvent is removed. The Tg is a characteristic property of PLGA, depending on composition, i.e. lactide to glycolide ratio, and molecular weight. Also, with non-crosslinked PLGA, Tg will decrease as degradation and moisture content increase in a body environment after implantation. In such instances, Tg often drops below body temperature (plasticization), converting the glassy solid microparticle of prior art non-crosslinked PLGA in vivo to a viscous liquid micro-droplet, which can dramatically accelerate the degradation kinetics and drug release rate in an uncontrolled manner. For organogel microparticles without oil, the PLGA matrix would be a glassy solid once the solvent is fully removed. Like non-crosslinked PLGA, crosslinked PLGA would be subject to gradual plasticization in physiological conditions. Unlike non-crosslinked PLGA, however, crosslinked PLGA will transform into a rubber above Tg, rather than a viscous liquid, which may manifest significant differences in drug release kinetics compared to non-crosslinked PLGA microparticles. For organogel microparticles containing an oil in a crosslinked polymer, the oil will act as a plasticizer, lowering the Tg and yielding a rubbery microparticle in its initial state. Therefore, the Tg may no longer be a primary rate controlling property for release of the active agent. It is believed that in such embodiments, plasticization in vivo will have a small or negligible effect on drug release, since no transition involving Tg will occur.

In certain embodiments, the biodegradable microparticles can comprise a blend of microparticles having different particle sizes and/or having a different polymer, and/or including different active agents. For example, blends of microparticles with different sizes and/or polymers of different molecular weight can be used to control the active agent release kinetics to give the desired release over time. Blends of microparticles with different active agents can be used for simultaneous multiple drug application, or for co-releasing therapeutically and diagnostically active agents from the same microparticle mixture.

According to the present invention, the composition of the biodegradable microparticles may be designed as desired for the intended use and therapeutic application. In an embodiment, the microparticles comprise from 5-99 wt.-%, 5-90 wt.-%, 10-70 wt.-%, 10-60 wt.-%, 15-50 wt.-%, or 15-35 wt. % of the covalently crosslinked polymer, or 5 to 95 wt.-%, 10-95 wt.-%, 40-95 wt.-%, 50-90 wt.-%, 60-90 wt.-%, or 60-85 wt.-%; and from 1 to 70 wt.-% of the active agent, or 5-65 wt.-%, 5-50 wt.-%, 10-45 wt.-%, or 10-45 wt.-%; wherein all weight percentages are selected to amount to 100% in total, and the wt.-% is based on the total mass of the microparticles. In embodiments where the microparticles comprise an organogel additionally including an oil, the amount of oil may be in the range from 1 to 70 wt.-% or 5-65 wt.-%, 5-60 wt.-%, 10-50 wt.-%, 10-40 wt.-%, 15-40 wt.-%, or 15-35 wt, wherein all weight percentages are selected to amount to 100% in total, and the wt.-% is based on the total mass of the microparticles.

High drug loads can be achieved in microparticles of embodiments of this invention. In one embodiment, the biodegradable microparticles have a drug load (active agent content) of at least about 5 wt.-%, at least about 10 wt.-%, at least about 20 wt.-%, at least about 30 wt.-%, at least about 35 wt.-%, at least about 40 wt.-%, at least about 45 wt.-%, at least about 50 wt.-%, at least about 55 wt.-%, at least about 60 wt.-%, at least about 65 wt.-%, at least about 70 wt.-%, or up to about 80 wt.-% (including any range of any of these values), based on the total mass of the microparticles. In an aspect thereof, the microparticles comprise from about 30 wt.-% to about 60 wt.-%, such as about 40 wt.-% to about 55 wt.-%, such as from about 45 wt.-% to about 50 wt.-% active agent, based on the total mass of the microparticles. For example, for a drug in the form of an oil such as travoprost, the drug load of the microparticles can be up to about 50 wt.-%, such as about 10 wt.-% to about 45 wt.-%, or about 45 wt.-%, based on the total mass of the microparticles. For drugs such as dexamathasone, the drug load of the microparticles can be up to about 70 wt.-%, such as up to about 65 wt.-%, or about about 40 wt.-% to about 60 wt.-%, based on the total mass of the microparticles. Highly potent active agents may be included with lower drug loads, such as about 5 wt.-% to about 20 wt.-% or even lower, such as about 1 wt.-% to about 5 wt.-%, based on the total mass of the microparticles.

In one embodiment, the mass ratio of the active agent and the polymer in the microparticles is about 3:1 to about 1:3, or about 2:1 to about 1:2, or about 1:1.

In one embodiment, the biodegradable microparticles are incorporated into a hydrogel, organogel or xerogel to form a sustained release drug-delivery system for use as an implant. In an aspect thereof, the content of the biodegradable microparticles with respect to the total weight of the implant is about 10 wt.-% to about 35 wt.-%, or about 23 wt.-% to about 27 wt.-%, or about 12 wt.-% to about 17 wt.-%, or about 30 wt.-% to about 35 wt.-%, or about 25 wt.-%, or about 15 wt.-%, or about 34 wt.-%.

If an organogel is used for incorporating the biodegradable microparticles, the organogel may comprise from about 1 wt.-% to about 90 wt.-% of a hydrophobic organic liquid or oil, or about 5 wt.-% to about 90 wt.-%, about 5 wt.-% to about 60 wt.-%, about 10 wt.-% to about 50 wt.-%, about 10 wt.-% to about 40 wt.-%, about 15 wt.-% to about 40 wt.-%, about 15 wt.-% to about 35 wt. %; or from about 5 wt.-% to about 95 wt.-% of the covalently crosslinked polymer gel matrix, or about 10 wt.-% to about 95 wt.-%, about 40 wt.-% to about 95 wt.-%, about 50 wt.-% to about-90 wt.-%, about 60 wt.-% to about 90 wt.-%, or from 60 wt.-% to about 85 wt.-%; and from about 1 wt.-% to about 50 wt.-% of the biodegradable microparticles, or about 5 wt.-% to about 50 wt.-%, about 5 wt.-% to about 40 wt.-%, about 10 wt.-% to about 30 wt.-%, or about 10 wt.-% to about 25 wt.-%; wherein all weight percentages are selected to amount to 100% in total, and the wt.-% is based on the total dry weight of the drug-delivery system or implant, respectively.

### Biodegradable Polymer

The biodegradable microparticles of certain embodiments of the present invention encapsulating the active agent include covalently and three dimensionally crosslinked polymers. The biodegradable polymer or polymer units in precursors thereof may be selected from, e.g., any of biodegradable natural, semisynthetic, synthetic, or biosynthetic polymer, or combinations thereof.

Natural polymers may include glycosaminoglycans, polysaccharides (e.g., dextran), polyaminoacids and proteins or mixtures or combinations thereof. Semisynthetic polymers may be selected from carboxymethyl celluloses, or alkyl celluloses such as methyl cellulose (MC), ethyl cellulose (EC).

In some aspects, synthetic precursors are utilized. Synthetic refers to a molecule not found in nature or not normally found in a human. Synthetic polymer may generally be any polymer that is synthetically produced by different types of polymerizations, including free radical polymerization, anionic or cationic polymerization, chain-growth or addition polymerization, condensation polymerization, ring-opening polymerization etc. The polymerization may be initiated by certain initiators, by light and/or heat, and may be mediated by catalysts.

Generally, the biodegradable microparticles of certain embodiments of the present invention encapsulating the active agent include covalently and three dimensionally crosslinked homopolymers or copolymers that can be selected from polyethylene glycol (PEG), polypropyleneglycol (PPG), polyvinyl alcohol, poly(vinylpyrrolidone), polylactic acid (PLA), polyglycolic acid (PGA), polylactic-co-glycolic acid (PLGA), poly-p-dioxanone, poly(trimethylene carbonate), polycaprolactone, and/or polyvinyl alcohol, random or block copolymers or combinations or mixtures of any of these, or one or more of polyaminoacids, glycosaminoglycans, polysaccharides, or proteins.

In a first embodiment, the biodegradable microparticles include at least one of crosslinked polylactic acid (PLA), polyglycolic acid (PGA), polylactic-co-glycolic acid (PLGA), poly(vinylpyrrolidone), poly-p-dioxanone, poly(trimethylene carbonate), polycaprolactone, and/or polyvinyl alcohol, or copolymers of any of these, and the at least one active agent. In particular embodiments thereof, the covalently crosslinked biodegradable polymer used in the microparticles of the present invention is one of crosslinked polylactic acid (PLA), or crosslinked polylactic-co-glycolic acid (PLGA).

In a second embodiment, the biodegradable microparticles include an organogel comprising at least one crosslinked polymer selected from polyethylene glycol (PEG), polypropyleneglycol (PPG), polyvinyl alcohol, poly(vinylpyrrolidone), polylactic acid (PLA), polyglycolic acid (PGA), polylactic-co-glycolic acid (PLGA), poly-p-dioxanone, poly(trimethylene carbonate), polycaprolactone, and/or polyvinyl alcohol, random or block copolymers or combinations or mixtures of any of these, or one or more of polyaminoacids, glycosaminoglycans, polysaccharides, or proteins, at least one oil, and the at least one active agent. In particular embodiments thereof, the covalently crosslinked biodegradable polymer used is one of crosslinked polyethylene glycol (PEG) or polypropyleneglycol (PPG), or crosslinked polylactic-co-glycolic acid (PLGA), or crosslinked copolymers of PEG and PLGA. In some aspects thereof, the active agent may be an oil itself, forming microparticles in the form of an organogel with the crosslinked polymer.

In other embodiments of the second embodiment, the biodegradable microparticles include an organogel comprising at least one crosslinked polymer selected from polylactic acid (PLA), polyglycolic acid (PGA), polylactic-co-glycolic acid (PLGA), poly(vinylpyrrolidone), poly-p-dioxanone, poly(trimethylene carbonate), polycaprolactone, and/or polyvinyl alcohol, or copolymers of any of these, at least one oil, and the at least one active agent. In particular embodiments thereof, the covalently crosslinked biodegradable polymer used is one of crosslinked polylactic acid (PLA), or crosslinked polylactic-co-glycolic acid (PLGA). In some aspects thereof, the active agent may be an oil itself, forming microparticles in the form of an organogel with the crosslinked polymer.

In other embodiments, copolymers of PEG and PLGA, particularly block copolymers of multiarm PEG copolymerized with PLGA may also be used in a similar manner.

Hydrophilic polymers may be selected from polyglycolic acid (PGA), and at least one of polyethylene glycol or polypropylene glycol may also be used. In one embodiment, the hydrophilic polymers comprise polyglycolic acid (PGA) or polyethylene glycol.

In a further embodiment of the invention, the covalently crosslinked polymer of the microparticles comprises a combination of a plurality of hydrophobic polymer units selected from at least one of polylactic acid (PLA) and polylactic-co-glycolic acid (PLGA), and a plurality of at least one of hydrophilic polymer units selected from at least one of polyethylene glycol (PEG) units, polypropylene glycol (PPG), or polyglycolic acid (PGA) units. In one embodiment the hydrophilic polymer units comprise polyethylene glycol (PEG) units.

In an embodiment, the crosslinked polymer of the microparticles is formed from multi-arm precursors including a combination of polylactic-co-glycolic acid (PLGA) units and polyethylene glycol (PEG) units. The ratio of polylactic-co-glycolic acid (PLGA) units to polyethylene glycol (PEG) units can be selected to be about 2.5:1 to about 1:2.5, or about 2:1 to about 1:2, or about 1:1.

In embodiments where PLGA is used, the polylactic-co-glycolic acid (PLGA) units can have an L/G ratio (in % L or G units) ranging from 0:100 to 100:0, or about 1:99 to about 99:1, or about 10:90 to about 90:10, or about 25:75 to about 75:25, or about 50:50.

In certain embodiments, in the biodegradable microparticles the polymer is covalently crosslinked by hydrolysable bonds between polymer units, which facilitates biodegradation in aqueous environments such as the human or animal body in vivo.

The hydrolysable bonds can include bonds or linkages selected from the group consisting of amine, amide, urethane, ester, anhydride, ether, acetal, ketal, nitrile, isonitrile, isothiocyanate, or imine bonds, and combinations thereof. These bonds are typically formed by condensation polymerization reactions of suitably functionalized gelators or precursors, respectively.

### Precursor Components

In embodiments of the invention, the covalently crosslinked polymer is formed by chemically covalently crosslinking multifunctional precursors. The precursors are, in one aspect, functionalized monomeric, oligomeric or polymeric molecules, bearing functional groups capable of crosslinking with other precursors or small molecule crosslinkers. A small molecule precursor generally refers to a precursor that is less than about 2000 Daltons. Examples for small molecule crosslinkers are diamine, triamine or tetramine compounds, di or tri isocyanates, etc. Non-limiting examples include ethylene diamine, tris(2-aminoethyl)amine (TAEA), or trilysine. The precursors, as well as the small molecule crosslinkers, can be linear or non-linear, such as branched, star-shaped, comb-shaped, or dendrimers, etc.

In an embodiment, the at least one precursor or small molecule crosslinker has a functionality for chemical crosslinking of greater than 2, such as 3 to 10, or 3 to 9, or 4 to 8, or 4. In one aspect, at least one of the precursors or small molecule crosslinkers has a functionality of equal to or greater than 3 in order to create a three-dimensional (3D) polymer network. Such a precursor can be non-linear, branched, star or comb-shaped, or a dendrimer. Thus, if linear difunctional polymer precursors are used in an embodiment, the small molecule crosslinker or the second polymer crosslinker is at least trifunctional, so that three dimensionally crosslinking can occur, forming a polymer matrix including the active agent and optionally the oil. When difunctional small molecule crosslinkers are used, the at least one multi-arm polymer precursor should have a functionality of at 3 or more, to achieve three dimensional crosslinking of the polymer matrix.

In an embodiment, the at least one precursor is a star shaped, multi-arm or dendrimer precursor having a core and from 2 to 10 arms, or 3 to 10 arms, 4 to 8 arms, or 4 or 6 arms, each arm comprising a polymer unit and having a terminus. The polymer units may comprise one or more of polyalkylene oxides such as polyethylene glycol, polypropylene glycol, poly(ethylene glycol)-block-poly(propylene glycol) copolymers, poloxamers such as commercially available Tetronic®, or commercially available Jeffamine® polymers, polyethylene oxide, polypropylene oxide; polyvinyl acetate, polyvinyl alcohol, poly(vinylpyrrolidone), polylactic acid (PLA), polyglycolic acid (PGA), polylactic-co-glycolic acid (PLGA), poly-p-dioxanone, poly(trimethylene carbonate), polycaprolactone, and/or polyvinyl alcohol, or copolymers of any of these, random or block copolymers or combinations or mixtures of any of these, or one or more units of polyaminoacids, glycosaminoglycans, polysaccharides, or proteins, while this list is not intended to be limiting.

The biodegradable microparticles comprising covalently crosslinked polymer may be formed of a plurality of hydrophobic polymer units, or from a plurality of hydrophilic polymer units, or a combination of hydrophobic and hydrophilic units. The polymer units may be selected to tailor the hydrophobicity and hydrophilicity of the microparticles in order to adjust it to the properties of the active agent. This adjustment enables control of certain aspects of release kinetics and degradation behavior of the microparticles.

In an embodiment of the invention, multi-arm precursors for hydrophobic biodegradable polymer units may include at least one of polylactic acid (PLA), and polylactic-co-glycolic acid (PLGA) units. The polymer units are suitably functionalized at their termini with the desired reactive groups, and the molecular weight of PLA or PLGA, or the L/G ratio in the PLGA copolymer may be varied according to desired polymer properties, such as hydrophobicity. Polycaprolactone, polyvinyl alcohol, or poly(vinylpyrrolidone) may also be used.

In the first embodiment as mentioned above, the biodegradable microparticles comprise a covalently crosslinked polymer including the active agent, and the polymer matrix or network is formed from at least one covalently crosslinkable precursor that is miscible with and/or soluble in a solvent. The precursors include polymer units of polylactic acid (PLA), polyglycolic acid (PGA), polylactic-co-glycolic acid (PLGA), poly(vinylpyrrolidone), polycaprolactone and/or polyvinyl alcohol. The active agent may be soluble in the same solvent, or dispersed therein in particulate form.

In the second embodiments as mentioned above, where the biodegradable microparticles comprise an organogel including oil (as an additive in addition to the active agent or the active agent being an oil), the polymer matrix of the organogel is formed from at least one covalently crosslinkable precursor comprising the polymer units as defined herein that is miscible with the oil, preferably soluble or dispersible in the oil, or optionally a mixture of the oil and a solvent.

In some embodiments, the microparticles comprise a polymer network or matrix, optionally in the form of an organogel, comprising or formed of at least one covalently crosslinked multi-arm precursor and a small molecule crosslinker. In other embodiments of the invention, the microparticles comprise a polymer network or matrix, optionally in the form of an organogel, comprising or formed of at least two covalently crosslinked multi-arm precursors.

Thus, a precursor is always a “functional polymer” or “functional material” such as a crosslinker (e.g., small molecule with low molecular weight) that is able to participate in the crosslinking reaction with another precursor to form a covalently crosslinked polymer network (or matrix). Thus, the term “non-functional polymer” refers to a polymer that may be present in the organogel of the present invention but does not participate in the crosslinking reaction with the precursors to form a polymer network. Precursors are chosen in consideration of the properties that are desired for the resultant microparticle polymer and in light of the structure at the time of formation, e.g., for compatibility with organic solvents if the crosslinked matrix is formed as an organogel.

The precursors used in the invention may include any polymer units described herein above as long as it is able to react with another precursor or crosslinker, in the presence of the active agent and optionally an oil, and forms a biocompatible and biodegradable crosslinked polymer in the form of microparticles.

Crosslinked polymer matrices for the biodegradable microparticles may be formed from any of the biodegradable polymers mentioned in the section above, and precursors to be crosslinked covalently include units of these polymers that bear functional groups capable of chemically crosslinking the precursors by forming covalent bonds, for forming a crosslinked polymer matrix.

In some aspects of the invention, at least one crosslinkable precursor is either hydrophobic or hydrophilic, and when two precursors are used, both may be hydrophobic, or both may be hydrophilic, or one is hydrophobic and the other one is hydrophilic. With more than two precursors, any mixture of hydrophilic and hydrophobic precursors may be chosen, depending on the desired properties of the microparticles. In addition, the precursors can be copolymers, incorporating both hydrophobic and hydrophilic substructures.

### Functional Groups for Crosslinking

The precursors have pairs of functional groups that react with each other, i.e., a first functional group on a first precursor capable of reacting with a second functional group on a second precursor or crosslinker. In an embodiment, a first multi-arm precursor comprising a first functional group, and a second multi-arm precursor or small molecule crosslinker comprising a second functional group are reacted, the functional groups being located at the terminus of the arms of the precursor or crosslinker, wherein the first or second functional group may be directly grafted to the precursor terminus, or via a linker molecule. The functional groups are capable to react with each other and form a covalent bond or linkage, for example, in electrophile-nucleophile reactions, or are configured to participate in other chemical crosslinking reactions as described below.

In certain embodiments of the invention, the first functional group and the second functional group are selected from an electrophile and a nucleophile, functional groups for click chemistry, functional groups for cycloadditions, particularly 1,3 dipolar cycloadditions, hetero-Diels-Alder cycloadditions, functional groups for nucleophilic ring openings, functional groups for non-aldol type carbonyl reactions, functional groups for addition reactions to carbon-carbon multiple bonds, polymerizable vinyl groups, or combinations thereof. The skilled person will know that certain pairs of functional groups may be classified in more than one of these groups. For example, in click chemistry, an azide reacting with dibenzocyclooctyne may be also seen as an electrophile-nucleophile reaction pair.

Thus, in one embodiment, the first functional group may be a nucleophile and the second functional group may be an electrophile, or vice versa, and the reaction between the first functional group and second functional group is an electrophile-nucleophile reaction that forms a covalent bond. According to certain embodiments of the invention, each precursor or crosslinker comprises at least two, or at least three terminal nucleophilic groups, or at least two, or at least three terminal electrophilic groups.

Nucleophiles may be selected from one of an amine such as a primary amine, a hydroxyl, a thiol, a carboxyl, a dibenzocyclooctyne, or a hydrazide group. In certain embodiments, at least one precursor comprises a nucleophile, such as a primary amine.

Electrophiles that can be used for the present invention may be selected from activated ester groups such as succinimidyl esters, succinimidyl carbonates; nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halogen. These electrophiles comprise functional groups that participate in the electrophile-nucleophile reaction and crosslink the precursors, and they preferably additionally include reactive groups that include hydrolysable groups or bonds, such as glutarate. For example, in an embodiment of the invention, a succinimidyl ester may comprise a reactive group such as succinimidyl succinate (SS), succinimidyl glutarate (SG), succinimidyl adipate (SAP), succinimidyl azelate (SAZ), or succinimidyl glutaramide. Such electrophile-nucleophile crosslinking reactions for multi-arm PEG-precursors are for example described in US 2002/0042473A1, which is incorporated by reference.

Thus, in an embodiment, the first functional and the second functional group are selected from functional group pairs for click chemistry, forming a covalent bond. For example, with azide and dibenzocyclooctyne functionalization, precursors may be crosslinked via so-called click-chemistry reactions. An overview of such types of reaction is given in H. C. Kolb; M. G. Finn; K. B. Sharpless (2001). “Click Chemistry: Diverse Chemical Function from a Few Good Reactions”, Angewandte Chemie International Edition, 40 (11): 2004-2021), incorporated herein by reference.

The functional group pairs for click chemistry can be selected functional groups for cycloadditions, particularly 1,3 dipolar cycloadditions, [3+2] cycloadditions such as alkene-nitrone cycloadditions or alkyne-nitrone cycloadditions, [4+2] cycloadditions, hetero-Diels-Alder cycloadditions; functional groups for thiol-ene reactions; functional groups for nucleophilic ring openings; functional groups for non-aldol type carbonyl reactions; functional groups for addition reactions to carbon-carbon multiple bonds; functional groups for Michael-type additions.

For example, the first functional group is an alkyne compound such as a dibenzocyclooctyne (DBCO), or a bicyclo[6.1.0]-nonyne (BCN); or a norbornene, or a trans-cyclooctene (TCO); and the second functional group is an azide, a 3,4 dihydroxyphenylacetic acid (DHPA), or a tetrazine (Tz). In these embodiments, the DBCO, BCN, norbornene, TCO, azide, DHPA and Tz functional groups are grafted to the termini of the multi-arm precursor via a linker such as an acid group, a diacid group, a functionalized aliphatic, heteroaliphatic, or aromatic or heteroaromatic group.

In another embodiment the first and second functional groups are selected for a [3+2] cycloaddition reaction such as alkene-nitrone cycloadditions or alkyne-nitrone cycloadditions. In a further embodiment, the first and second functional group are selected for a [4+2] cycloaddition reaction, particularly a hetero Diels-Alder reaction, wherein the first functional group is an aldehyde or imine compound, and the second functional group is a 1,3 diene compound, an unsaturated carbonyl compound, or a nitroso-alkene compound. In still another embodiment, the first and second functional group are selected for a thiol-ene reaction, wherein the first functional group is a thiol compound and the second functional group is an alkene, preferably a terminal alkene. In a further embodiment, the first and second functional group are selected for nucleophilic ring openings, wherein the first functional group is selected from an epoxide, thiirane, aziridine, or lactam, and the second functional group is nucleophile as mentioned above. In another embodiment, the first and second functional group are selected for non-aldol type carbonyl reactions, wherein the first functional group is an aldehyde or ketone compound, and the second functional group is a primary amine, a hydrazide, acyl hydrazide or aminooxy compound, to form an imine, amide, isourea, hydrazone, acyl hydrazone or oxime linkage.

In still another embodiment, the first functional and the second functional group are selected from functional groups that may be radically polymerized/crosslinked.

In such embodiments, the first functional group and the second functional group are selected e.g., from polymerizable vinyl groups and acrylates such as (meth)acrylic acid, (meth)acrylic acid esters, acrylamides, fumaric acid, maleic acid and combinations thereof. Crosslinking is induced thermally or photochemically, optionally with the use of initiators such as photo initiators such as free radical photo initiators (Norish I type such as 2,2-dimethoxy-1,2-diphenyl-ethan-1-one, 2-Hydroxy-2-methyl-1-phenylpropanone, 1-hydroxy-cyclohexylphenylketone; or Norish II type such as benzophenone and its derivative and isopropyl thioxanthone in combination with a synergist such as tertiary amines2-ethylhexyl-(4-N,N-dimethyl amino)benzoate, and 2-ethyl-(4-N,N-dimethylamino)benzoate); or cationic photo-initiators.

Such crosslinking mechanisms with terminal vinyl group functionalized precursors are for example described in US 2021/0251893A1, which is incorporated herein by reference.

### Multi-Arm Precursors

The term “multi-arm” precursor means that the precursors are branched, i.e. non-linear. In the case of a multi-arm polymer, a core refers to a contiguous portion of a molecule joined to polymer unit arms that extend from the core, with the arms having a nucleophile or electrophile functional group, which is often at the terminus of the branch. Precursors may have, e.g., 2-100 arms, with each arm having a terminus, bearing in mind that some precursors may be dendrimers or other highly branched materials such as dendrimers. An arm on a precursor refers to a linear chain of chemical groups, i.e. a polymer unit as defined herein, that connect a cross linkable functional group to a polymer core. Some embodiments are precursors with between 3 and 300 arms; artisans will immediately appreciate that all the ranges and values within the explicitly stated ranges are contemplated, e.g., 4, 6, 8, 10, 12, 4 to 16, 8 to 100, 6, 8, 10, 12, or at least 4 arms.

In certain embodiments, the multi-arm precursor of the invention has a core and from 2 to 10 arms, or 3 to 10 arms, 4 to 8 arms, or 4 or 8 arms, each arm comprising a polymer unit and having a terminus, the terminus bearing a functional group for crosslinking as defined above. Since in multi-arm precursors the arms extend from the central core, their polymer terminus is identical and all arms can favourably be functionalized in one reaction with identical functional groups.

Each of the polymer units in a multi-arm precursor may have an average molecular weight (Mw) e.g., in the range from about 1,000 to about 100,000 Daltons, or from about 10,000 to about 60,000 Daltons, or from about 15,000 to about 50,000 Daltons.

### Core

The core of a multi-arm precursor is a structure appropriate to provide the desired number of arms of the precursor. For example, for 4-arm polymer units and precursors, the core can be a pentaerythritol or ethylenediamine structure, whereas for 8-arm polymer units and precursors, the core can be a hexaglycerol structure.

As disclosed above, in certain embodiments, the polymeric network of the biodegradable microparticle is formed from at least two precursors, at least one of them being a multi-arm precursor, the second one being a small molecule crosslinker or also a multi-arm precursor. The first multi-arm precursor comprising first functional groups, and a second precursor selected from a small molecule crosslinker or a multi-arm precursor comprising second functional groups, the functional groups being located at the terminus of the arms or the molecule. In various embodiments of the invention, each of the first functional group and the second functional group is selected from a group of crosslinkable functional groups as defined above. In some embodiments, the functional groups are selected from an electrophile and a nucleophile, and the reaction between the first functional group and second functional group is an electrophile-nucleophile reaction or polycondensation that forms the covalent bonds in the polymer of the biodegradable microparticles.

In some embodiments, when each precursor is multi-arm, it comprises two or more arms and thus, for example, two or more same or different electrophiles or nucleophiles, or any of the other above pairs of crosslinkable first and second functional groups, such that each nucleophile may react with another electrophile (within the same precursor or another precursor) in an electrophilic-nucleophilic reaction to form a crosslinked polymeric product. Thus, for example, in some aspects, the precursor has at least 4 arms, at least 8 arms, or at least 10 arms, wherein each arm terminates with either a nucleophile or an electrophile that may or may not be the same as its other arms.

In one embodiment, the biodegradable microparticle comprises at least two multi-arm precursors comprising a first multi-arm precursor comprising a nucleophile, and a second multi-arm precursor comprising an electrophile. In this embodiment, the first multi-arm precursor and the second multi-arm precursor are covalently cross-linked with each other in an electrophile-nucleophile reaction. In this context, the multi-arm refers to at least 4 arms, at least 8 arms, such as at least 10 arms. In this embodiment, the nucleophile can be an amine such as a primary amine, a thiol, a dibenzocyclooctyne, or a hydrazide, and the electrophiles can be succinimidyl esters, succinimidyl carbonates, nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halides. For example, in an embodiment of the invention, a succinimidyl ester may comprise a reactive group such as succinimidyl succinate (SS), succinimidyl glutarate (SG), succinimidyl adipate (SAP), succinimidyl azelate (SAZ), or succinimidyl glutaramide.

Some precursors may have a longer hydrolysis half-life as compared to others. This means that the time required for them to degrade may be longer. This may, in part, be due to the reactive group comprised in that precursor. For example, a PLPGA polymer comprising an electrophile group such as a succinimidyl ester group that comprises a reactive group such as a succinimidyl succinate (SS) has a shorter hydrolysis half-life as compared to a PLGA polymer comprising an electrophile group such as a succinimidyl ester group that comprises a reactive group such as a succinimidyl glutarate (SG).

In one embodiment, the biodegradable microparticle comprises two multi-arm precursors and it may comprise a first multi-arm precursor comprising a nucleophile such as an amine, and a second multi-arm precursor comprising an electrophile such as a succinimidyl ester. In another embodiment, the biodegradable microparticle crosslinked polymer may comprise a first multi-arm precursor comprising a nucleophile such as an amine such as a primary amine, and a second multi-arm precursor comprising an electrophile such as a succinimidyl ester comprising a first reactive group. In this embodiment, the reactive group is selected from succinimidyl succinate (SS), succinimidyl glutarate (SG), succinimidyl adipate (SAP) or a succinimidyl azelate (SAZ).

As disclosed above, the polymeric network of the biodegradable microparticle is formed from at least two precursors, at least one of them being a multi-arm precursor, the second one being a small molecule crosslinker or also a multi-arm precursor. The first multi-arm precursor comprising first functional groups, and a second precursor selected from a small molecule crosslinker or a multi-arm precursor comprising second functional groups, the functional groups being located at the terminus of the arms or the molecule. In various embodiments of the invention, each of the first functional group and the second functional group is selected from a group of crosslinkable functional groups as defined above. In some embodiments, the functional groups are selected from an electrophile and a nucleophile, and the reaction between the first functional group and second functional group is an electrophile-nucleophile reaction or polycondensation that forms the covalent bonds in the polymer of the biodegradable microparticles.

### PLA/PLGA Precursors

In certain embodiments, precursors are polylactic-co-glycolic acid (PLGA) precursors, i.e. having PLGA polymer units at the core of the multi-arm precursor.

In an embodiment, such precursors may have the following exemplary structure with a pentaerythritol derived core of a rather hydrophobic and oil soluble 4a20K PLGA-NHS:

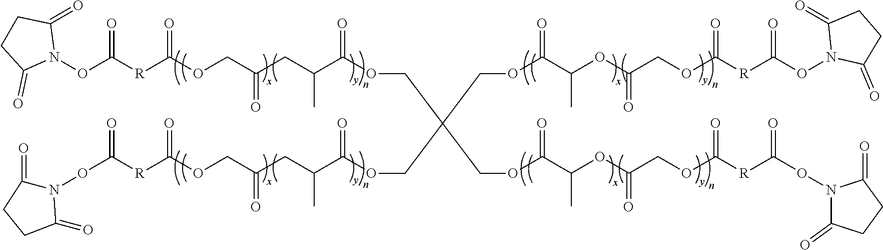

According to its designation, this is a 4 arm PLGA with each PLGA unit having a Mn of about 5,000 Daltons, and the PLGA units have an L/G ratio of 50:50 (i.e., 1:1), R together with the two carbonyl groups to which it is bound is part of a diacid linker derived from a saturated or unsaturated biocompatible organic diacid, such as one of oxalic acid, malonic acid, succinic acid, glutaric acid, adipic acid, maleic acid, fumaric acid, and NHS designates a N-hydroxy succinimide electrophile as the functional group on each arm's terminus. x is an integer and define the number of lactic acid units and y is an integer defining the number of glycolic acid units in the PLGA molecule. For 50:50 PLGA x and y are equal. n is an integer defining the number of PLGA blocks in an optional block copolymer unit, for 50:50 PLGA n is 1.

Another example of an electrophile functionalized 4-arm PLGA precursor is 4a20K PLGA5050-SAP-NHS (x and y are about 15):

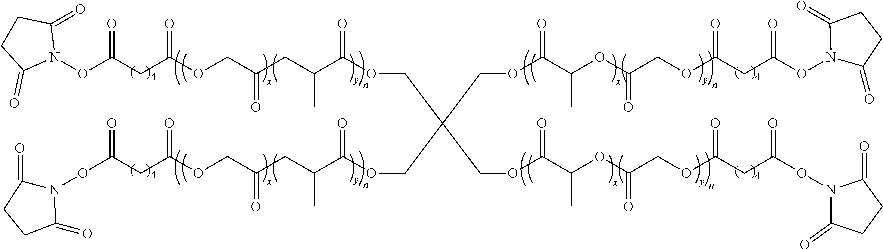

In other embodiments, the multi-arm PLGA precursor may also be derived from ethylenediamine as the core instead of pentaerythritol. Using different cores, the precursor may have other than 4 arms, such as 6, 8, 10 or 12 arms, up to dendrimer compounds having up to 100 or mor arms.

In various embodiments of the invention, the biodegradable microparticle crosslinked polymer comprises or is built from one multi-arm precursor that comprises polymer units of polylactic-co-glycolic acid (PLGA) units, in other embodiments polylactic acid (PLA) units, or combinations or (block) copolymers thereof. In such embodiments the biodegradable microparticles are formed using at least one crosslinker, preferably a small molecule amine such as tris(2-aminoethyl)amine (TAEA), which is oil soluble, or trilysine.

In certain embodiments, polylactic acid (PLA) units are preferred.

In some embodiments, these PLGA and/or PLA based microparticles may include an oil, forming an organogel as the biodegradable microparticle.

In some embodiments of the invention, the biodegradable microparticle crosslinked polymer comprises at least one multi-arm precursor that comprises hydrophobic polymer units selected from polylactic acid (PLA) units and polylactic-co-glycolic acid (PLGA) units, or combinations or (block) copolymers thereof, and at least one further multi-arm precursor comprising hydrophilic polymer units, preferably selected from polyethylene glycol (PEG) and polyglycolic acid (PGA).

In certain embodiments of the present invention, PLA and/or PLGA units used in the precursors have an average molecular weight in the range from about 1,000 to about 100,000 Daltons, or in a range from about 10,000 to about 60,000 Daltons, or in a range from about 15,000 to about 50,000 Daltons. In some embodiments, the PLA and/or PLGA units have an average molecular weight in a range from about 10,000 to about 40,000 Daltons, or of about 20,000 Daltons. PLA and/or PLGA precursors of the same average molecular weight may be used, or PLA and/or PLGA precursors of different average molecular weight may be combined with each other. The average molecular weight of the PLA and/or PLGA precursors used in the present invention is given as the number average molecular weight (Mn), which, in certain embodiments, may be determined by gel permeation chromatography against polystyrene standard according to standardized methods.

Precursors as mentioned above are commercially available, such as 4arm PLA SS (20k), 4arm PLGA 50:50 SS (20k, 60k), or 4arm PLGA 75:25 SS (20k, 60k) and others can be obtained from Nanosoft Polymers, Winston-Salem, US, or other vendors such as Creative PEGWorks, Chapel Hill, NC, USA, SinoPEG, CN, or Akina Inc., West Lafayette, Indiana, USA.

### PEG Precursors

In some embodiments, precursors are polyethylene glycol precursors, i.e. having polyethylene polymer units at the core of the multi-arm precursor. Thus, in some embodiments, the microparticles' polymer network of covalently cross-linked precursors is made of or includes at least one polyethylene glycol-containing precursor. Polyethylene glycol (PEG, also referred to as polyethylene oxide) refers to a polymer with a repeat group (CH₂CH₂O) n, with n being at least 3.

A polymeric precursor having a polyethylene glycol thus has at least three of these repeat groups connected to each other in a linear series. A PEG polymer that terminates in a hydroxyl group or a methoxy group that does not participate in the cross-linking reaction between the precursors is referred to as a “non-functional PEG” described herein above and thus, not used as one of the precursors. Thus, a PEG polymer that terminates in a nucleophile selected from a primary amine, a thiol, a dibenzocyclooctyne, or a hydrazide is considered as a “functional PEG” and can be used as one of the precursors. Further, a PEG polymer that terminates in an electrophile selected from succinimidyl esters, succinimidyl carbonates, nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halides is considered as a “functional PEG” and can be used as one of the precursors.

The polymer network of the biodegradable microparticle drug-delivery system of the present invention may comprise one or more multi-arm PEG precursors having from 2 to 10 arms, or 4 to 8 arms, or 4, 5, 6, 7 or 8 arms. It has to be noted that since multi-arm precursors have a core, a 2-arm PEG precursor, for example, differs from simple linear PEG by the presence of the core structure. Two arm precursors can form 3D crosslinked networks with crosslinkers having a functionality of at least 3. The PEG precursors may have a different or the same number of arms. In certain embodiments, the PEG precursors used in the organogel of the present invention have 4 and/or 8 arms. In certain embodiments, a combination of 4- and 8-arm PEG precursors is utilized.

In certain embodiments of the present invention, polyethylene glycol units used as precursors have an average molecular weight in the range from about 1,000 to about 100,000 Daltons, or in a range from about 10,000 to about 60,000 Daltons, or in a range from about 15,000 to about 50,000 Daltons. In some embodiments the polyethylene glycol units have an average molecular weight in a range from about 10,000 to about 40,000 Daltons, or of about 20,000 Daltons. PEG precursors of the same average molecular weight may be used, or PEG precursors of different average molecular weight may be combined with each other. The average molecular weight of the PEG precursors used in the present invention is given as the number average molecular weight (Mn), which, in certain embodiments, may be determined by gel permeation chromatography against polystyrene standard according to standardized methods.

In a 4-arm PEG, each of the arms may have an average arm length (or molecular weight) of the total molecular weight of the PEG divided by 4. A 4a20kPEG precursor, which is one precursor that can be utilized in the present invention thus has 4 arms with an average molecular weight of about 5,000 Daltons each. An 8a20k PEG precursor, which may be used in addition to the 4a20kPEG precursor in the present invention, thus has 8 arms each having an average molecular weight of 2,500 Daltons. Accordingly, a 4a20K PLGA precursor has 4 arms with an average molecular weight of about 5,000 Daltons each.

In general, when referring to a polymer precursor having a certain average molecular weight, such as a 15kPEG- or a 20kPLGA-precursor, the indicated average molecular weight (i.e., a Mn of 15,000 or 20,000, respectively) refers to the polymer unit part of the precursor, before end groups are added (“20k” here means 20,000 Daltons, and “15k” means 15,000 Daltons—the same abbreviation is used herein for other average molecular weights of PEG or other polymer precursors). In certain embodiments, the Mn of the polymer unit part of the precursor is determined by gel permeation chromatography against polystyrene standard according to standardized methods. The degree of substitution with end groups as disclosed herein may be determined by means of H-NMR after end group functionalization.

In various embodiments of the invention, the biodegradable microparticle comprises at least two multi-arm precursors, the first precursor is a multi-arm PEG precursor comprising a nucleophile such as an amine, such as a primary amine. In some of these embodiments, the second multi-arm precursor is a multi-arm PEG precursor comprising an electrophile such as a succinimidyl ester. In other of these embodiments, the second multi-arm precursor is a multi-arm PLGA precursor comprising an electrophile such as a succinimidyl ester.

In some embodiments of the invention, the biodegradable microparticle crosslinked polymer comprises three multi-arm precursors, the first multi-arm precursor is a multi-arm PEG precursor comprising a nucleophile such as an amine, such as a primary amine. In this embodiment, the second multi-arm precursor is a multi-arm PEG precursor comprising an electrophile such as a succinimidyl ester comprising a first reactive group. In this embodiment, the third multi-arm precursor is a multi-arm PEG precursor comprising an electrophile such as a succinimidyl ester comprising a second reactive group. In this embodiment, the first and the second reactive groups can be selected from succinimidyl succinate (SS), succinimidyl glutarate (SG), succinimidyl adipate (SAP) or a succinimidyl azelate (SAZ). SS, SG, SAP and SAZ are all functionalization linkers attached to the polymer comprising reactive groups composed of N-succinimidyl esters of the corresponding diacids, that have an ester group connection to the polymer at the second acid of the diacid that can degrade by hydrolysis in water. In some embodiments, the first multi-arm precursor is succinimidyl succinate (SS) and the second multi-arm precursor is succinimidyl glutarate (SG).

Each and any combination of electrophilic- and nucleophilic-group containing PEG precursors disclosed herein may be used for preparing the implant according to the present invention. For example, any 4-arm or 8-arm PEG precursor (e.g., having a succinimidyl ester comprising a SS, SG, SAP, or SAZ reactive group) may be combined with any 4-arm or 8-arm PEG precursor (e.g., having a NH₂ group or another nucleophile). Furthermore, the PEG units of the electrophile- and the nucleophile group-containing precursors may have the same or may have a different average molecular weight.

One such combination is a PEG amine precursor and two PEG succinimidyl ester precursors, one comprising an SS reactive group and another comprising an SG reactive group. In certain embodiments, the inventors have found that by keeping the molar ratio of PEG amine to PEG succinimidyl ester at about 1:1 and by varying the molar ratio of the reactive groups of the succinimidyl esters SS and SG, the time taken by the polymeric network to degrade in an aqueous solution under physiological conditions can be controlled, although other ratios are contemplated. The amount of PEG SS and SG to be used to reach a particular molar ratio of the two reactive groups can be calculated by a skilled artisan and described as follows.

The amount of PEG amine and PEG esters (SS and SG) to be used is calculated through stoichiometric equations of molar proportion and converting moles to grams. First, the reactive end group molar ratio between the amine, the succinimidyl succinate, and succinimidyl glutarate is determined. In an example formulation, 4a20k PEG NH₂, 4a20k PEG SS, and 4a40k PEG SG are used. The molar ratio between amine and succinimidyl ester groups is about 1:1, and the molar ratio between SS and SG is about 80:20. The final end group molar ratio between the 4a20k NH₂:4a20k SS:4a40k SG is about 1.0:0.8:0.2. Next, gram to mole stoichiometric conversions, and vice versa, are used to determine mass amounts. Below outlines an example calculation of 4a20k SS at the molar ratios above with 100 g of 4a20k NH₂:

$$100 g 4 a 20 k PEG NH 2 \times \frac{1 mol PEG NH 2}{20000 g PEG NH 2} \times \frac{4 mol NH 2}{1 mol PEG NH 2} \times \frac{0.8 mol SS}{1 mol NH 2} \times \frac{1 mol PEG SS}{4 mol SS} \times \frac{20000 g PEG SS}{1 mol PEG SS} = 80 g 4 a 20 k PEG SS$$

Alternatively, the amounts of PEGs can be determined by calculating the “molecular weight between crosslinks” (MWc) and the arm length ratio. The MWc can be calculated through the sum of the average arm length of each multi-arm PEG precursor.

$$PEG Arm Length = \frac{PEG MW}{\# arms} \times PEG molar ratio$$

$$MWc = PEG NH 2 Arm Length + PEG SS Arm Length + PEG SG Arm Length$$

The arm length ratio is calculated by dividing the PEG Arm Length over the MWc. By multiplying the arm length ratio for a particular multi-arm precursor with a total PEG batch size, the amount of multi-arm precursor can be determined. Below outlines an example calculation for the amount of 4a20k PEG SS with a total batch size of 100 g PEG:

$$PEG NH 2 Arm Length = \frac{20000 Da}{4} \times 1.0 = 5000 Da$$

$$PEG SS Arm Length = \frac{20000 Da}{4} \times 0.8 = 4000 Da$$

$$PEG SG Arm Length = \frac{40000 Da}{4} \times 0.2 = 2000 Da$$

$$MWc = 5000 Da + 4000 Da + 2000 Da = 11000 Da$$

$$PEG SS Arm Length Ratio = \frac{4000 Da}{11000 Da} = 0.3 \; 64$$

$$Mass PEG SS = 0.364 \times 100 g = 36.4 g$$

Similar calculations can be done for other types of polymers as described herein.

In certain embodiments, 4-arm PEGs with an average molecular weight of about 20,000 Daltons and 4-arm PEGs with an average molecular weight of about 40,000 Daltons can be used for forming the covalently crosslinked polymer of the microparticles according to the present invention.

Thus, the first precursor, and/or the second precursor may be a 4a20k precursor, wherein 4 denotes the arms and 20k denotes the Mn. Thus, for example, the first, second and/or the third precursor may be a 4a40k precursor. Thus, for example, the first and/or the second precursor may be a 4a20k precursor and the third precursor may be a 4a40k precursor.

### Active Agents:

The active agent in the biodegradable microparticles of embodiments of the invention can be a therapeutically active agent or a diagnostically active agent, or combinations thereof. It may be a single active agent or a plurality of active agents.

Therapeutically active agents may be steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, viruses, viruses for gene delivery such as AAV, etc., or any combinations thereof.

In some embodiments, steroids may be corticosteroids that can comprise hydrocortisone, loteprednol, cortisol, cortisone, prednisolone, methylprednisolone, dexamethasone, betamethasone, triamcinolone, aldosterone or fludrocortisone.

In some embodiments, NSAIDs can comprise diclofenac (e.g., diclofenac sodium), flurbiprofen (e.g., flurbiprofen sodium), ketorolac (e.g., ketorolac tromethamine), bromfenac, or nepafenac.

In some embodiments, IOP lowering agents and/or glaucoma medications can comprise prostaglandin analogs (e.g., bimatoprost, latanoprost, travoprost, or latanoprostene bunod), rho kinase inhibitor (e.g., netarsudil), adrenergic agonists (epinephrine or dipivefrin), beta-adrenergic antagonists also known as beta blockers (e.g., timolol, levobunolol, metipranolol, carteolol, or betaxolol), alpha2-adrenergic agonists (e.g., apraclonidine, brimonidine, or brimonidine tartrate), carbonic anhydrase inhibitors (e.g., brinzolamide, dichlorphenamide, methazolamide acetazolamide, acetazolamide, or dorzolamide), pilocarpine, echothiophate, demercarium, physostigmine, and/or isofluorophate.

In some embodiments, anti-infective can comprise antibiotics comprising ciprofloxacin, tobramycin, erythromycin, ofloxacin, gentamicin, fluoroquinolone antibiotics, moxifloxacin, and/or gatifloxacin; antivirals comprising ganciclovir, idoxuridine, vidarabine, and/or trifluridine; and/or antifungals comprising amphotericin B, natamycin, voriconazole, fluconazole, miconazole, clotrimazole, ketoconazole, posaconazole, echinocandin, caspofungin, and/or micafungin.

In some embodiments, antimetabolites can comprise methotrexate, mycophenolate, or azathioprine.

In some embodiments, antifibrotic agents can comprise mitomycin C or 5-fluorouracil.

In some embodiments, angiogenesis inhibitors can comprise anti-VEGF agents (e.g., aflibercept, ranibizumab, bevacizumab), PDGF-B inhibitors (e.g., Fovista®), complement antagonists or inhibitors (e.g., eculizumab, avacincaptad pegol), tyrosine kinase inhibitors (e.g., sunitinib, axitinib), and/or integrin antagonists (e.g., natalizumab and vedolizumab).

In some embodiments, cytoprotective agents can comprise ebselen, sulforaphane, oltipraz or dimethyl fumarate.

In some embodiments, neuroprotective agents can comprise ursodiol, memantine or acetylcysteine.

In some embodiments, anaesthetic agents can comprise lidocaine, proparacaine or bupivacaine.

In some embodiments, the active agent can be dexamethasone, ketorolac, diclofenac, vancomycin, moxifloxacin, gatifloxicin, besifloxacin, travoprost, 5-fluorouracil, methotrexate, mitomycin C, prednisolone, bevacizumab (Avastin®), ranibizumab (Lucentis®), sunitinib, pegaptanib (Macugen®), timolol, latanoprost, brimonidine, nepafenac, bromfenac, triamcinolone, difluprednate, fluocinolide, aflibercept, or combinations thereof. In some embodiments, the agent may be dexamethasone, ketorolac, diclofenac, moxifloxacin, travoprost, 5-fluorouracil, or methotrexate. In some embodiments, the agent is dexamethasone. In some embodiments, the agent is ketorolac. In some embodiments, the agent is travoprost.

Diagnostically active agents may be, e.g., imaging agents, markers, or visualization agents. Generally, diagnostic agents may be substances used to examine the body in order to detect impairment of its normal functions. In some cases, diagnostic agents may be agents with a functional purpose, such as for use in the detection of ocular deformities, ailments, and pathophysiological aspects. For example, the diagnostic agent may be an important and effective diagnostic adjuvant, such as a dye (e.g., fluorescein dye, indocyanine green, trypan blue, a dark quencher such as a cyanine dye, an azo dye, an acridine, a fluorene, an oxazine, a phenanthridine, a naphthalimide, a rhodamine, a benzopyrone, a perylene, a benzanthrone, pra benzoxanthrone), to aid in visualization of ocular tissues. The diagnostic agent may comprise paramagnetic molecules, fluorescent compounds, magnetic molecules, radionuclides, x-ray imaging agents, and/or contrast media. In some embodiments, a diagnostic agent may include radiopharmaceuticals, contrast agents for use in imaging techniques, allergen extracts, activated charcoal, different testing strips (e.g., cholesterol, ethanol, and glucose), pregnancy test, breath test with urea 13C, and various stains/markers. In some embodiments, the labelling moiety is a fluorescent dye or a dark quencher, selected from the group consisting of a coumarin, a cyanine dye, an azo dye, an acridine, a fluorene, an oxazine, a phenanthridine, a naphthalimide, a rhodamine, a benzopyrone, a perylene, a benzanthrone, and a benzoxanthrone. In particular non-limiting embodiments, the fluorescent dye is or is the residue of a compound selected from the group consisting of Coumarin, Fluorescein, Cyanine 3 (Cy3), Cyanine 5 (Cy5), Cyanine 7 (Cy7), Alexa dyes, bodipy derivatives, (E)-2-(4-(phenyldiazenyl) phenoxy) acetic acid, 3-(3′,3′-dimethyl-6-nitrospiro[chromene-2,2′-indolin]-1′-yl) propanoate (Spiropyran), 3,5-dihydroxy benzoate and (E)-2-(4-(phenyldiazenyl) phenoxy) acetic acid, or combinations thereof.

In certain embodiments of the invention, the active agent is dispersed, embedded or encapsulated in the covalently crosslinked biodegradable polymer. In certain embodiments, the active agent may be in particulate form.

In certain embodiments of the invention, the active agent is a drug in the form of a liquid oil at temperatures up to 37° C., e.g., travoprost, etc., which forms an organogel with the crosslinked biodegradable polymer of the microparticles, that in vivo includes the active agent as a liquid in the crosslinked polymer. According to other embodiments of the invention the active agent may be oil soluble and dissolved in a hydrophobic organic liquid, or oil, respectively, which forms an organogel with the crosslinked biodegradable polymer of the microparticles. Alternatively, the active agent is oil insoluble and can be dispersed in particle form in a hydrophobic organic liquid or oil, or emulgated in liquid form, and also forming an organogel with the crosslinked biodegradable polymer of the microparticles. In these embodiments, the biodegradable microparticles are formed of an organogel including the active agent as or in a liquid phase immobilized in the crosslinked particle.

In embodiments where the active agent is used in particulate form, the active agent particles may be micronized particles, e.g., having a D50 particle size of less than about 15 μm, or less than 10 μm and/or a D99 particle size of less than about 100 μm, or less than about 50 μm, or a D90 particle size of about 50 μm or less, or 5 μm or less and/or a D98 particle size of about 10 μm or less. In other embodiments the active agent particles may be nanosized particles, e.g., having a D50 particle size of less than about 100 nm, or less than about 50 nm, and/or a D99 particle size of less than about 50 nm, or a D90 particle size of about 5 nm or less and/or a D98 particle size of about 10 nm or less. Particle sizes are determined as disclosed in the “Definitions” section herein.

### Oil/Additives

In certain embodiments of the invention, an oil is included in the crosslinked polymer, forming an organogel that constitutes the microparticles of these embodiments. These organogel microparticles mostly have a rubbery appearance. The oil, in general a hydrophobic organic liquid, can be used to modify the release of an active agent from the microparticle drug-delivery system. One or more of its properties such as hydrophobicity, viscosity, compatibility with the active agent, solubility or insolubility of the active agent in the oil, and the like can be suitably selected to control the release of the active agent from the organogel-microparticles of these embodiments. For example, when the biodegradable microparticles for sustained release drug-delivery of embodiments of the invention are used in a hydrogel or other matrix as an implant inserted into the human body, or are directly used as an oral dosage form, the oil can diffuse out of the organogel microparticles into the aqueous environment together with the active agent dissolved therein, before or concurrently with a diffusion of the active agent out of the oil. If the active agent is, for example, a water-soluble solid particulate dispersed in the hydrophobic organic liquid, the oil can be used to slow down contact of the aqueous environment with the active agent and to delay leaching out of the active agent from the organogel microparticles.

In certain embodiments, the oil or hydrophobic organic liquid is liquid at human body temperature, such as a temperature of about 37° C. or lower, or liquid in the range of 0° C. to 40° C., or 10° C. to 38° C., or 15° C. to 37° C., or 25° C. to 37° C., or at 37° C. The term “liquid” may include viscous fluids having a creamy or wax-like but non-solid appearance. Also, for some hydrophobic organic liquids that undergo hydration in aqueous embodiments such as body fluids the melting point at a certain temperature may be different for the hydrated material than for the non-hydrated. In certain embodiments of the present invention, the hydrated form of such materials are liquid under those conditions as described above.

In an embodiment the active agent is dissolved or dispersed in the oil before being incorporated into the crosslinked polymer of the microparticles. In another embodiment, the active agent is itself an oil or oil-like hydrophobic organic liquid or forms at least a part thereof. An example is travoprost as the active agent.

In certain embodiments, the oil may include an oil mixture. The oil may be a biocompatible vegetable oil, a synthetic oil or a mineral oil, a liquid fatty acid or triglyceride composition, or it may be a hydrophobic biodegradable liquid polymer, or combinations thereof.

In certain aspects of the present disclosure the oil is a biocompatible oil that may be selected from the group comprising triethyl citrate, acetyl triethyl citrate (ATEC), acetyl tributyl citrate (ATBC), α-tocopherol (vitamin E), α-tocopherol acetate; plant or vegetable oils such as sesame oil, olive oil, soybean oil, sunflower oil, coconut oil, canola oil, rapeseed oil, nut oils such as hazelnut, walnut, pecan, almond, cottonseed oil, corn oil, safflower oil, linseed oil, etc., ethyl oleate, castor oil and derivatives thereof (Cremophor®), lipids being liquid at 37° C. or lower, such as saturated or unsaturated fatty acids, monoglycerides, diglycerides, triglycerides (Myglyols®), phospholipids, glycerophospholipids, sphingolipids, sterols, prenols, polyketides, hydrophobic biodegradable liquid polymers, low melting point waxes such as plant, animal or synthetic waxes, lanolin, jojoba oil, or combinations thereof.

In certain aspects, the oil is liquid at human body temperature, and may have a glass transition temperature and/or a melting temperature equal to or below 45° C., or equal to or below 37° C.

In certain embodiments, the oil is non-volatile at 37° C. and ambient pressure, non-toxic, and/or biocompatible, and/or capable of being cleared from an implantation site, metabolized and/or cleared unchanged from the body.

### Method of Manufacture

Methods for manufacturing polymer microparticles are known to the skilled artisan, and these methods can be principally applied and suitably adapted in embodiments of the present invention.

In certain embodiments of the invention, a method for manufacturing the biodegradable microparticles for sustained release drug-delivery involves a method selected from one of the following methods: emulsion solvent evaporation-extraction, emulsion solvent diffusion, supercritical fluid emulsion, coacervation, spray drying, hydrogel template, use of microfluidic systems, membrane extrusion emulsification, particle replication in non-wetting templates (PRINT) technique, electro hydrodynamic atomization (EHDA) or electro-spraying, or the microparticles may be obtained from the gas saturated solutions (PGSS) method, or by 3D printing.

Lagreca et al., Progress in Biomaterials, 2020, volume 9, pages 153-174, incorporated herein by reference, provides an overview over these preparation methods for microparticles made of PLA or PLGA polymers. These methods can be principally used with other biodegradable polymers and the multi-arm precursors as described herein as well.

Exemplary preparation methods that may be favorably used in embodiments of the present invention are the single emulsion technique and the double emulsion technique.

Functionalized precursors of hydrophobic polymer units such as polylactic acid (PLA), polylactic-co-glycolic acid (PLGA), poly(vinylpyrrolidone), or polycaprolactone and a lipophilic drug are dissolved in an organic, non-polar solvent and combined with crosslinkers, optionally in a second solution, and the combined solutions are added under agitation, stirring, sonication or homogenization to a water phase including a surfactant or emulsifying agent, thereby forming a oil-in-water type emulsion. The microparticles are formed and hardened during the elimination of the organic solvent, for example by evaporation, and crosslinking. The evaporation can be facilitated by continuous stirring or using a negative pressure solvent draw system.

A variation of this technique for active agents/drugs that are solid and/or insoluble in the organic solvent comprises dispersing or suspending the active agent/drug in the organic solvent comprising the dissolved polymer precursors or the crosslinker solution. Optionally, an oil may be added, resulting in organogel microparticles.

Applying the double emulsion technique, the at least one suitably functionalized polymer precursor is dissolved in an organic solvent, and an aqueous solution containing a water-soluble drug and optionally a hydrophilic crosslinker or second functionalized precursor is added to this organic solution and the mixture emulsified into a water-in-oil type emulsion, for example by a sonicator. The obtained emulsion is then added to a large volume of a continuous water phase containing an emulsifying agent, thereby forming a double emulsion (water-in-oil-in-water type). The solidification and crosslinking of the microparticles takes place during emulsion formation and subsequent solvent removal.

More complex techniques like the microfluidic technology rely on the same basic principle as the single or double emulsion techniques with the use of corresponding microfluidic apparatus equipment and are also suitable to produce the inventive microparticles. The preparation of non-crosslinked monodispersed biodegradable polymer microparticles using a microfluidic flow-focusing device has been described, for example, in Xu, Q., et al., “Preparation of Monodispersed Biodegradable Polymer Microparticles Using a Microfluidic Flow-Focusing Device for Controlled Drug Delivery”, Small, Vol 5 (13): 1575-1581, 2009. The use of microfluidic devices to generate microspheres has further been described by Duncanson, W. J. et al., “Microfluidic Synthesis of Monodisperse Porous Microspheres with Size-tunable Pores”, Soft Matter, Vol 8, 10636-10640, 2012, and U.S. Pat. No. 8,916,196 B1 describes an apparatus and method for the production of emulsion based microparticles that can be used in connection with the present invention.

In a generally applicable embodiment, the method for manufacturing the biodegradable microparticles for sustained release drug-delivery comprising the steps of (1) forming a gel comprising a covalently crosslinked polymer in the presence of at least one active agent and optionally at least one oil and optionally a first solvent, (2) producing microparticles wherein the at least one active agent is dispersed within the covalently crosslinked polymer, and, (3) optionally removing solvent.

When oil is present in step (1), an organogel is formed, that includes the oil in the crosslinked polymer from which the microparticles are formed. The active agent may be dissolved or dispersed in the oil.

In one embodiment, the method comprises the steps of: (a) dissolving at least one of the polymer precursors in a first solvent, producing a first mixture; (b) providing a second mixture comprising a crosslinker in a second solvent; (c) adding at least one active agent, and optionally an oil, to at least one of the first mixture or second mixture; (d) combining the first mixture and the second mixture to produce a first phase; (e) providing a second phase comprising a third solvent, that is immiscible with the first and second solvents; (f) introducing, under agitation, the first phase into the second phase, thereby producing an emulsion of dispersed first phase in the second phase; and (g) removing the first, second solvent and/or third solvent. Agitation includes stirring, sonication, vortexing, or by using homogenizers as known in the art.

In certain embodiments, the step of producing microparticles (step (2)) or step (f) comprises forcing the first phase through a mesh or injecting the first phase into the agitated second phase, the first and/or second solvent and/or third solvent optionally comprising additives such as emulsifiers, surfactants, dispersing adjuvants, or porogens, to form microspheric or nanospheric particles.

In certain embodiments of the method, the first solvent and/or second solvent is an organic solvent wherein the precursors and crosslinkers are soluble, and the second solvent may be the same as the first solvent. The third solvent is a solvent wherein the precursors, crosslinkers and/or the formed organogel is insoluble.

The first solvent and/or second solvent is selected from acetone, acetonitrile, benzyl alcohol, chloroform, dichloromethane (DCM), dioxane, dimethyl carbonate (DMC), DMSO, ethanol, ethyl acetate, ethyl formate, ethyl propionate, glycofurol, hexafluoro-isopropanol, isosorbide dimethyl ether, isopropanol, methyl chloride, methylene chloride, methyl ethyl ketone, N-methylpyrrolidone, propylene carbonate, or tetrahydrofuran, or any mixtures thereof, and the third solvent is water, an alcohol such as methanol, ethanol or propanol, or any mixture thereof.

The additives can be surfactants or emulsifiers such as polyvinyl alcohol (PVA), polyethylene glycol sorbitan monolaurate (Tween®), sorbitan monolaurate (Span®), sodium dodecyl sulfate (SDS), and may be used in the second, aqueous phase; and/or porogens such as inorganic salts (NaCl, KCl, sodium or potassium carbonates or bicarbonates, ammonium bicarbonate), Pluronics; sodium or potassium oleate; gelatin; mustard oil, mineral oil; cyclodextrins; carbohydrates, bovine serum albumin (BSA); photo initiators, radical polymerization initiators, and combinations thereof.

According to some embodiments, method steps 1 and 2 make use of oil-in-water emulsion or water-in-oil single or double emulsion technology, or combinations thereof, particularly single or double emulsion technique, or microfluidic technology, or combinations thereof.

Removal of the first and/or second and/or third solvent is done by one of hot air convection or direct drying, indirect or contact drying, spray drying, dielectric drying, vacuum drying, freeze drying, supercritical or superheated steam drying, or combinations of any of these.

In an exemplary embodiment, active agent containing biodegradable microparticles for drug delivery are prepared by an oil-in-water emulsification solvent evaporation/extraction technique. A suitably functionalized multi-arm precursor as described herein, for example a 4a20kPLGA-NHS and the at least one active agent, for example travoprost, are dissolved in a first solvent, for example dichloromethane (DCM) to prepare a single-phase solution or, when the agent is in particulate form and insoluble in the solvent a suspension, which is the first solution (or suspension). A second solution is prepared, comprising the small molecule crosslinker or a second functionalized multi-arm precursor in the same or a similar miscible solvent. Both solutions are combined and added as a dispersed phase (DP) into an agitated third solution comprising a non-miscible solvent, designated the continuous phase (CP).

For hydrophobic polymer precursors such as PLA or PLGA, the solvent of the first and second solution is non-polar, and the solvent in the third solution is polar and non-miscible with the first solvent, and vice versa for hydrophilic polymer precursors. Adding the dispersed phase of combined first and second solution to the agitated third solution can for example be done by injection via a syringe or syringe pump. If hydrophobic precursors and nonpolar solvents are used for the combined dispersed phase, the third solution can be, for example, an aqueous solution of polyvinyl alcohol (PVA) forming the continuous phase (CP). The solution may have a concentration of about 1% (w/w) or any other suitable concentration. PVA acts mainly as an emulsifier or surfactant stabilising the microdroplets of the DP that subsequently crosslink and harden into microspheres, but also increases the viscosity of the continuous phase which helps forming spheres.

In certain embodiments, the injection occurs immediately prior to passing through an in-line homogenizer to disperse the DP into nascent microparticles. The addition of the dispersed phase to the continuous phase at this introduction stage allows time to disperse the droplets before crosslinking and hardening to create the primary emulsion. These nascent microparticles in the CP stream can then flow into a stirring CP (quench medium) in a jacketed reactor maintained at a controlled temperature. This emulsion is stirred for a sufficient period of time in the quench medium to extract and evaporate DCM and harden the microparticles.

In certain embodiments, the resultant microparticles are filtered off, washed and sieved to the appropriate size fraction, for example using a vibratory sieve agitator. Optionally, the microparticles are dried or lyophilized to remove residual solvent and to finally yield the dry biodegradable microparticles comprising covalently crosslinked polymer. Since the crosslinked polymer microparticles of the embodiments of the present invention are rather heat stable, removal of the solvents can advantageously be done by one of hot air convection or direct drying, indirect or contact drying, spray drying, dielectric drying, vacuum drying, supercritical or superheated steam drying, or combinations of any of these, which may use elevated temperatures, so that complex and expensive lyophilization steps or freeze-drying can be avoided.

In one embodiment, when all components have been combined in the reaction mixture in step (f), the at least one precursor and one small molecule crosslinker or the at least two precursors react in an electrophile-nucleophile reaction to form a covalently cross-linked matrix. If oil is present, organogel microparticles are formed. The reaction may be initiated or promoted by heating, or can occur at ambient conditions.

In another embodiment, when all components have been combined in the reaction mixture in step (f), the at least one precursor and one small molecule crosslinker or the at least two precursors that are functionalized by polymerizable acryl groups react in a radical polymerization reaction, optionally photoinduced, to form a covalently cross-linked polymer matrix in microparticulate form. The reaction may be initiated or promoted by heating, or can occur at ambient conditions. Click chemistry functionalization as described above may be used as well.

An emulsion such as formed in step (f) with the precursors mixed therein can be made with viscosity suitable for introduction through a small gauge needle using manual force. A small gauge needle has a diameter less than the diameter of a needle with a gauge of 27, e.g., 28, 29, 30, 31, 32, or 33 gauge, with the gauge being specific for inner and/or outer diameters. Thus a viscosity of between about 1 to about 100,000 mPa's may be used; artisans will immediately appreciate that all the ranges and values within the explicitly stated ranges are contemplated e.g., about 10 to about 10,000 mPas, less than about 5 to about 10,000 mPas, less than about 100 or about 500 mPas, or between about 1 and about 100 mPa·s. The viscosity may be controlled, e.g., by choosing appropriate precursors, adjusting solids and or solvent concentrations, and reaction kinetics. In general, lower concentrations of precursors, increased hydrophilicity, lower molecular weights favor a lower viscosity.

### Release Kinetics

In embodiments of the invention, the biodegradable microparticles allow for drug-delivery allow to modify or tailor the release of an active agent from the microparticles by several measures. For example, tailoring or suitably selecting the precursor components forming the crosslinked polymer of the microparticles according to their hydrophilic and/or hydrophobic properties has an influence on active agent release. Furthermore, if an oil containing organogel type polymer matrix is used, the diffusion kinetics and release of an active agent from the microparticles or drug delivery system including them can be modified or controlled by suitably selecting the oil component according to its properties such as one or more of hydrophobicity, viscosity, compatibility with the active agent, solubility or insolubility of the active agent in the oil, etc.

Accordingly, in various embodiments of the present invention the selection of the oil, and/or the type, constitution and properties such as hydrophobicity of the polymeric network, and/or the L/G ratio in PLCA, may be used to tune the release rate. Each of these individual parameters can be selected alone or in combination with each other to provide for the controlled release of the active agent.

In certain embodiments, the biodegradable microparticles and/or the drug-delivery system including them are formulated to make an active agent available over an extended period of time, thereby allowing a reduction in dosing frequency compared to an immediate release dosage form, such as for example a solution of an active agent that is topically applied onto the eye (i.e., eye drops). In certain embodiments, the release of the active agent comprises constant active agent release, tapered active agent release as well as any combination thereof such as a constant active agent release followed by a tapered active agent release. The “sustained release” may be measured in vitro in an aqueous solution under physiological conditions such as at pH 7.2-7.4 and 37° C. and is considered to be the same or substantially the same when the drug-delivery system is administered in vivo to a subject.

In various embodiments of the present invention the active agent release follows zero order release kinetics or substantially zero order release kinetics, preferably without a “burst” of active agent at the beginning of the period. A burst is a rapid initial release of active agent from the microparticles within a relatively short interval directly after insertion of an implant, e.g. for the first day following insertion. According to the invention the burst is minimized.

Certain embodiments of the present invention may provide for a release of a therapeutically effective amount of the active agent for a period of time, such as up to 1 year, up to 9 months, up to 6 months, up to 3 months, up to 1 month, or up to about 25 days after administration. Other embodiments of the present invention may provide for a release of a therapeutically effective amount of the active agent of up to about 14 days, or up to about 21 days after administration, or a release of a therapeutically effective amount of the active agent for a period of about 6 hours or longer after administration, or for a period of about 12 hours, or 24 hours or longer or about 48 or longer, or about 72 hours or longer or about 7 days or longer, or about 10 days or longer after administration. The present invention contemplates all of the above lower and higher time periods in any combination of ranges.

In some embodiments of the present invention the crosslinked polymer biodegradable microparticles delay the release of a water-soluble active agent or accelerate the release of a hydrophobic active agent. In other embodiments the crosslinked polymer biodegradable microparticles delay the release of a hydrophobic active agent or accelerate the release of a water-soluble active agent.

In one aspect of the present invention, biodegradable microparticles for sustained drug delivery or a sustained release drug-delivery system including the biodegradable microparticles, such as a pharmaceutically acceptable implant are provided for a controlled release of the active agent comprised therein (e.g., the total amount). Throughout this disclosure, controlled release is to be considered as the controlled release measured from the time and under conditions wherein the implant is first immersed in an aqueous solution under physiological conditions such as at pH 7.2-7.4 and temperature 37° C. After exposure to physiological conditions, the crosslinked polymer or organogel of the microparticles or comprised in the drug-delivery system may slowly release the hydrophobic organic liquid from the organogel and concomitantly forms a hydrogel.

In certain embodiments, the controlled release can be characterized as the amount of the active agent released on day 1 is from 0 to 50% of the total amount of the active agent, the amount of the active agent released per day from day 2 until the last day of release is from 0 to 50% of the total amount of the active agent, and/or the number of days required for 100% release of the total amount of the active agent is at least 2 days.

In certain embodiments, the controlled release can be characterized as the amount of the active agent released on day 1 is from 0 to about 50% of the total amount of the active agent, the amount of the active agent released per day from day 2 until the last day of release is from 0 to about 50% of the total amount of the active agent, and/or the number of days required for 100% release of the total amount of the active agent is at least 3 days.

In certain embodiments, the controlled release can be characterized as the amount of the active agent released on day 1 is from 0 to about 50% of the total amount of the active agent, the amount of the active agent released per day from day 2 until the last day of release is from 0 to about 50% of the total amount of the active agent, and/or the number of days required for 100% release of the total amount of the active agent is at least 4-7 days.

In certain embodiments, the controlled release can be characterized as the amount of the active agent released on day 1 is from 0 to about 50% of the total amount of the active agent, the amount of the active agent released per day from day 2 until the last day of release is from 0 to about 50% of the total amount of the active agent, and/or, the number of days required for 100% release of the total amount of the active agent is at least 10-15 days.

In certain embodiments, the controlled release can be characterized as the amount of the active agent released on day 1 is from 0 to about 50% of the total amount of the active agent, the amount of the active agent released per day from day 2 until the last day of release is from 0 to about 50% of the total amount of the active agent, and/or the number of days required for 100% release of the total amount of the active agent is at least 10-30 days.

In certain embodiments, the controlled release can be characterized as the amount of the active agent released on day 1 is from 0 to about 50% of the total amount of the active agent, the amount of the active agent released per day from day 2 until the last day of release is from 0 to about 50% of the total amount of the active agent, and/or the number of days required for 100% release of the total amount of the active agent is greater than 30 days.

According to certain embodiments of the invention, the controlled release is characterized by: the amount of the active agent released on day 1 is from 0 to about 25%, 0 to about 20% 0 to about 10%, 0 to about 5%, or about 0% of the total amount of the active agent, the amount of the active agent released per day from day 2 until the last day of release is from 0 to about 50% or from 0 to about 40% or from 0 to about 30% or from 0 to about 20% or from 0 to about 10% or from 0 to about 5% of the total amount of the active agent. In certain embodiments, the number of days required for 100% release of the total amount of the active agent is at least 3 days but no greater than 30 days, 25 days, or no greater than 16 days. In other embodiments the time is as disclosed above.

In one embodiment, the controlled release characterized above comprises a zero-order release, such as near zero order release, or substantially zero order release. In one embodiment, the zero-order release or near zero order release or substantially zero order release begins at least 1 day after the pharmaceutically acceptable implant has been immersed under physiological conditions such as pH 7.2-7.4 and 37° C.

A dosage form or implant exhibiting zero order release rate would exhibit a relatively straight line in a graphical representation of percent active agent released versus time. In certain embodiments of the present invention, the zero-order release is accomplished over the entire period of release. In certain embodiments of the present invention, the zero-order release is accomplished over a part of the period of release. In certain such embodiments the zero-order release is accomplished from the end of day 1, i.e., from 24 hours after the start of the release, to the end of the release. If less or no release is accomplished before the end of day 1 such release would be considered to have a lag time for one day or 24 hours. Such a lag time could also be longer. If a high release is accomplished before the end of day 1 such release would be considered to have a burst during the first day or 24 hours. Such a burst time could also be longer. Zero order release can also be accomplished during the entire period of release. The entire period of release is, in this context, defined until 95% of the release is accomplished.

Zero order release is defined to be accomplished, within the meaning of the present invention, if during the respective time the release is proportional to elapsed time. Proportional to elapsed time means that the proportional release is calculated based on the entire time of the zero order release defining a straight line (release in % cumulative release during the entire period of time during which zero order is accomplished divided by said entire period of time defining a straight line) and the release at any time point in between, i.e., start of zero order release and end of zero order release is within 20% points of the % cumulative release of said proportional release defined by said straight line.

### Sustained Release Drug Delivery System:

In certain embodiments, a sustained release, biodegradable drug-delivery system is provided that comprises the biodegradable microparticles for sustained release drug-delivery as described herein. For providing the drug-delivery system the biodegradable microparticles are incorporated into a hydrogel, xerogel or organogel, optionally by using extrusion methods or 3D printing. In embodiments of the invention, such a system is used for coating a medical implant. In other embodiments, the drug-delivery system is used for producing or forming a medical implant, wherein the biodegradable microparticles are embedded or dispersed in a hydrogel, xerogel or organogel matrix.

Since the biodegradable microparticles are heat stable, rubbery materials that are above their glass transition temperature, unlike conventional non-crosslinked polymer particles, they do not melt and processing steps involving heat, for example extrusion methods can be used for shaping such medical implants or for applying implant coatings. Methods involving processing steps requiring application of heat can be, for example, extrusion methods such as hot melt extrusion, or injection molding of a reaction mixture comprising the biodegradable microparticles dispersed in a hydrogel, xerogel or organogel or precursors thereof, or 3D printing methods. In such methods, the gelling occurs before and/or during extrusion or injection molding of the gel-forming mass comprising the biodegradable microparticles. With extrusion methods being available for producing these implants, high throughput production is possible with the heat stable biodegradable microspheres.

In certain embodiments of the present invention, biodegradable microparticles, as defined herein, are incorporated, i.e. dispersed or distributed in a biodegradable hydrogel, organogel or xerogel. In a specific embodiment the biodegradable microparticles are uniformly dispersed in said biodegradable polymer. The preparation of a hydrogel matrix suitable for incorporating the biodegradable microparticles of the present invention is described in the following sections “PEG Hydrogel” and “Method of preparing drug delivery systems or implants comprising biodegradable microparticles”, and the described principles also apply to the incorporation of the biodegradable microparticles in hydrogels made of different polymers than PEG, such as those described herein as useful for the preparation of the microparticles themselves, as well as for organogel matrices (including an oil) instead of hydrogels, for Since the biodegradable microparticles are heat stable, rubbery materials that are above their glass transition temperature, unlike conventional non-crosslinked polymer particles, they do not melt and processing steps involving heat, for example extrusion methods can be used for shaping such medical implants or for applying implant coatings. Methods involving processing steps requiring application of heat can be, for example, extrusion methods such as hot melt extrusion, or injection molding of a reaction mixture comprising the biodegradable microparticles dispersed in a hydrogel, xerogel or organogel or precursors thereof, or 3D printing methods incorporating the biodegradable microparticles of the present invention.

### PEG Hydrogel

In certain embodiments, the hydrogel comprises a polymer network comprising one or more units of polyethylene glycol. In a certain embodiments of the present invention, the polymer network forming the hydrogel contains polyethylene glycol (PEG) units. PEGs are known in the art to form hydrogels when cross-linked, and these PEG hydrogels are suitable for pharmaceutical applications e.g. as matrix for drugs intended to be administered to all parts of the human or animal body.

The polymer network of the hydrogel implants of the present invention may comprise one or more multi-arm PEG units having from 2 to 10 arms, or 4 to 8 arms, or 4, 5, 6, 7 or 8 arms. In certain embodiments, the PEG units used in the hydrogel of the present invention have 8 arms. In certain particular embodiments, an 8-armed PEG is utilized.

In certain embodiments, the polyethylene glycol units are 4- to 10-arm polyethylene glycol units, or 8-arm polyethylene glycol units.

The molecular weight of the polyethylene glycol refers to a number average molecular weight (Mn) weight. Multi-arm PEG units with a specified molecular weight as used herein may be abbreviated in the form of e.g. 8a15kPEG, referring to an 8-arm PEG with a molecular weight of 15,000 Daltons, as also described herein above.

In a 4-arm PEG, each of the arms may have an average arm length (or molecular weight) of the total molecular weight of the PEG divided by 4. A 4a20kPEG precursor, which is a particularly suitably precursor for use in the present invention thus has 4 arms with an average molecular weight of about 5,000 Daltons each. An 8a20k PEG precursor, which could also be used in combination with or alternatively to the 4a20kPEG precursor in the present invention, thus has 8 arms each having an average molecular weight of 2,500 Daltons. Longer arms may provide increased flexibility as compared to shorter arms. PEGs with longer arms may swell more as compared to PEGs with shorter arms. A PEG with a lower number of arms also may swell more and may be more flexible than a PEG with a higher number of arms. In certain particular embodiments, only a 4-arm PEG precursor is utilized in the present invention. In certain particular embodiments, two different 4-arm PEG precursors are utilized in the present invention. In certain other embodiments, a combination of a 4-arm PEG precursor and an 8-arm precursor is utilized in the present invention. In addition, longer PEG arms have higher melting temperatures when dry, which may provide more dimensional stability during storage.

In certain embodiments, the polymer network of the hydrogel embedding the biodegradable microparticles is formed by reacting an electrophilic group-containing multi-arm-polymer precursor with a nucleophilic group-containing cross-linking agent. In particular certain embodiments wherein the multi-arm-polymer precursor is a 4- to 10-arm polyethylene glycol precursor, or an 8-arm polyethylene glycol precursor.

In certain embodiments, electrophilic end groups for use with PEG precursors for preparing the hydrogels of the present invention are N-hydroxysuccinimidyl (NHS) esters, including but not limited to NHS dicarboxylic acid esters such as the succinimidylmalonate group, succinimidylmaleate group, succinimidylfumarate group, “SAZ” referring to a succinimidylazelate end group, “SAP” referring to a succinimidyladipate end group, “SG” referring to a succinimidylglutarate end group, and “SS” referring to a succinimidylsuccinate end group.

In certain embodiments, the multi-arm polymer precursor is selected from the group consisting of 8-arm-15K-SG polyethylene glycol or 8-arm-15K-SAZ polyethylene glycol.

In certain embodiments, the electrophilic group is selected from the group consisting of a succinimidylglutarate (SG) group and a succinimidylazelate (SAZ) group.

In certain embodiments of the present invention, the polymer network is formed by reacting an electrophilic group-containing multi-arm-polymer precursor with a nucleophilic group-containing cross-linking agent, the electrophilic group is selected from the group consisting of a succinimidylglutarate (SG) group and a succinimidylazelate (SAZ) group, and the multi-arm polymer precursor is selected from the group consisting of 8-arm-15K-SG polyethylene glycol or 8-arm-15K-SAZ polyethylene glycol; and the nucleophilic group-containing cross-linking agent is a trilysine, or 8-arm-15K-SAZ polyethylene glycol; and the nucleophilic group-containing cross-linking agent is a trilysine, or the polymer network comprises 8-arm-polyethylene glycols being cross-linked including a group represented by the following formula

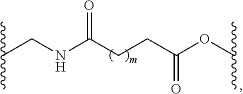

wherein m is 2 or 6.

Thus, in certain embodiments, the PEG-precursor is an NHS dicarboxylic acid ester-terminated multi-arm PEG precursor that can be represented by the formula:

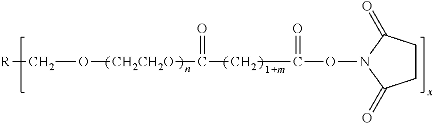

wherein n is determined by the molecular weight of the respective PEG-arm, m is an integer from 0 to 10, and specifically is 1, 2, 3, 4, 5, 6, 7, 8, 9, or 10, and x is the number of arms (and thus can e.g. 2, 4, 8, etc., see above). Where m is 1, each arm is terminated with a succinimidylsuccinate (SS) end group, where m is 2, each arm is terminated with a succinimidylglutarate (SG) group, where m is 3, each arm is terminated with a succinimidyladipate (SAP) group, and where m is 6, each arm is terminated with a succinimidylazelate (SAZ) group. With these specific electrophilic end groups, multi-arm PEG units may be abbreviated in the form of e.g. 4a20kPEG-SAP, referring to a 4-arm PEG with a succinimidyladipate end group and a molecular weight of 20,000. In the above formula, R is a core structure appropriate to provide the desired number of arms. For 4-arm PEG units and precursors, R can be a pentaerythritol structure, whereas for 8-arm PEG units and precursors, R can be a hexaglycerol structure.

In certain embodiments, the multi-arm-polymer precursor has a mass average molecular weight in the range from about 10,000 to about 20,000 Daltons. In a more specific certain embodiment, the multi-arm-polymer precursor has a mass average molecular weight of 15,000±10% Daltons.

In certain embodiments, reactions of e.g. nucleophilic group-containing crosslinkers and electrophilic group-containing PEG units, such as a reaction of amine group-containing crosslinkers with activated ester-group containing PEG units, result in a plurality of PEG units being crosslinked by the crosslinker via an amide group.

In the case of PEGs with NHS-ester end groups such as succinimidylazelate (SAZ)-, succinimidyladipate (SAP)- or succinimidylgluatarate—(SG)-terminated PEG units (see above), the reaction with amine group-containing crosslinkers result in a plurality of PEG units being crosslinked by the crosslinker via a hydrolyzable linker having the formula:

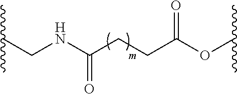

wherein m is an integer from 0 to 10, and specifically is 1, 2, 3, 4, 5, 6, 7, 8, 9, or 10. For a SAZ-end group, m would be 6. For a SAP-end group, m would be 3, for a SG-end group, In would be 2 and for an SS-end group, m would be 1.

In a certain embodiment, the nucleophilic group-containing crosslinking agent is an amine. In another embodiment, the nucleophilic group-containing crosslinking agent is a small molecule amine with a molecular weight below 1,000 Da, comprising two or more primary aliphatic amine groups. In a certain embodiment, the nucleophilic group-containing crosslinking agent is a small molecule amine selected from the group consisting of dilysine, trilysine, tetralysine, ethylenediamine, 1,3-diaminopropane, 1,3-di-aminopropane, diethylenetriamine, and trimethylhexamethylenediamine. In one embodiment, the nucleophilic group-containing crosslinking agent is a trilysine. In one specific embodiment, the nucleophilic group-containing crosslinking agent is trilysine acetate.

In another embodiment, the trilysine is labeled with a visualization agent selected from the group consisting of a fluorophore such as fluorescein, rhodamine, coumarin, and cyanine. Specifically, the nucleophilic group-containing crosslinking agent is fluorescein-conjugated trilysine. More specifically, the fluorescein-conjugated trilysine is obtained by reacting trilysine acetate with N-hydroxysuccinimide (NHS)-fluorescein. Even more specifically, wherein the trilysine is labeled by partial conjugation with the visualization agent.

### Method of Preparing Drug Delivery Systems or Implants Comprising Biodegradable Microparticles

In a further aspect, the present invention is directed to a method of manufacturing a sustained release drug delivery system, e.g. an implant, comprising the biodegradable microparticles for sustained drug-delivery as described herein. The method comprises the steps of:

a) preparing biodegradable microparticles for sustained drug-delivery as described herein,
b) preparing a precursor mixture containing hydrogel, organogel or xerogel precursors and the biodegradable microparticles,
c) crosslinking the precursor mixture using a cross-linker to form a polymer network and to obtain a hydrogel or organogel mixture comprising the polymer network, and
d) drying the hydrogel or organogel mixture to provide the drug delivery system or implant.

The types of components, the contents of the components and the mass ratios of the mass ratios described in the preceding sections on microparticle, and hydrogel manufacturing also apply correspondingly for the method of manufacturing of the present invention.

In another aspect, the present invention is directed to a sustained release biodegradable drug-delivery system or implant obtainable by the method as described above.

Steps b) and c) of the above method can be performed by any suitable mixing and crosslinking method as further described herein and as known for example from US 2021/0251893A1 or US 2018//085307A1. Mixing of components in syringes, strand or pellet extrusion of the implants, also for direct insertion into the human or animal body as known in the art can be used. Furthermore, since the biodegradable microparticles are heat stable, processing steps involving heat, for example extrusion methods can be used for shaping such medical implants or for applying implant coatings.

Methods involving processing steps requiring application of heat can be, for example, extrusion methods such as hot melt extrusion, or injection molding of a reaction mixture comprising the biodegradable microparticles dispersed in a hydrogel, xerogel or organogel or precursors thereof.

For example, for acrylate modified hydrogel or organogel precursors, 3D printing methods using radiation curing such as UV-curing may be employed, specifically if more complex implant structures are desired.

### Administration

The sustained release, biodegradable drug-delivery system comprising the biodegradable microparticles can be provided in the form of an implant as described above, such as a medical implant or a pharmaceutically acceptable implant, but also as an implant coating, or an oral dosage form, etc.

If the sustained release, biodegradable drug-delivery system comprising the biodegradable microparticles is an implant, the implant may be one of an intraocular implant, intracaveal implant, intracameral implant, an implant for introduction into the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrasceleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroid, tenon, cornea, cancer tissue, organ, prostate, breast, joint space, subdural, dental, subcutaneous, carpal tunnel, perivascular, surgically created space or injury, void space, and potential space.

In embodiments of certain embodiments of the invention, the biodegradable microparticles or the drug-delivery system comprising the biodegradable microparticles may be formulated for direct or indirect administration via diverse routes such as oral, parenteral, or by operative insertion or injection. Oral dosage forms may consist of the biodegradable microparticles of the present invention, which may optionally be enterically coated, or filled into capsules. Injectable formulations may consist of the biodegradable microparticles of the present invention suspended in an injectable liquid, and the like.

### Treatment Methods

According to the invention, the sustained release, the biodegradable microparticles or the biodegradable drug-delivery system comprising the biodegradable microparticles are configured for use as a medicament, such as for use in treating a disease or medical condition of a patient.

In an embodiment, the method for treating a disease or medical condition of a patient comprises administering the biodegradable microparticles comprising a therapeutically active agent or administering a hydrogel, organogel or xerogel comprising the biodegradable microparticles to the patient in order to release the active agent over an extended period of time.

A treatment method of an embodiment of the invention comprises incorporation of biodegradable microparticles into a hydrogel, organogel or xerogel, wherein forming a hydrogel, organogel or xerogel is done in situ at a treatment site of the patient in order to release the active agent over an extended period of time.

In another embodiment, a treatment method comprises incorporation of biodegradable microparticles into a hydrogel, organogel or xerogel, wherein the hydrogel, organogel or xerogel is prefabricated and delivered to or implanted at a treatment site the patient in order to release the active agent over an extended period of time.

The treatment site may be one of the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrasceleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroid, tenon, cornea, cancer tissue, organ, prostate, breast, joint space, subdural, dental, subcutaneous, carpal tunnel, perivascular, surgically created space or injury, void space, and potential space.

In embodiments of the invention, the disease or medical condition to be treated is an eye disease, such as back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy; or glaucoma, ocular hypertension, hyphema, presbyopia, cataract, retinal vein occlusion, inflammation. The ocular disease may be selected from retinal neovascularisation, choroidal neovascularisation, Wet AMD, Dry AMD, retinal vein occlusion, diabetic macular edema, retinal degeneration, corneal graft rejection, retinoblastoma, melanoma, glaucoma, autoimmune uveitis, uveitis, proliferative vitreoretinopathy, and corneal degeneration, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpiginous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angiitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, retinitis pigmentosa, Leber's Congenital Amaurosis, Choroideremia, X-linked retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, and red-green color blindness.

The methods described in this section can also comprise administration of the biodegradable microparticles, optionally included in a drug-delivery system such as a pharmaceutically acceptable implant, in combination with another agent, also termed “combination therapy”.

In one embodiment, the combination therapy comprises administering the biodegradable microparticles, optionally included in a drug-delivery system such as a pharmaceutically acceptable implant, in combination with one or more additional agents either on the same or different day. In one embodiment, the additional agent to be administered in a combination therapy can be a liquid formulation of the agent, or it may be comprised in an oral dosage form. Thus, the additional agent can be any small molecule, large molecule, a protein, a nanoparticle, or any other of the active agents described herein.

The method of treatment comprising administering the biodegradable microparticles, optionally included in a drug-delivery system such as a pharmaceutically acceptable implant, may comprise any one of intravitreal, intracameral, subconjunctival, retrobulbar, sub-tenon, subretinal, and suprachoroidal injections. The method of administration may also be topical or oral.

The active agent or the additional agent to be administered in a combination therapy, may also be a diagnostic agent. Diagnostic agents have been described above and may be substances used to examine the body in order to detect impairment of its normal functions. In some cases, diagnostic agents may be agents with a functional purpose, such as for use in the detection of ocular deformities, ailments, and pathophysiological aspects.

### Release Controlling Method

In one aspect, the invention relates to method for controlling the release of an active agent from a sustained release, biodegradable drug-delivery system as described herein before, including the biodegradable microparticles. Control of release may be done by either one or a combination of the following measures:
selecting the L/G ratio of the polylactic-co-glycolic acid (PLGA) units to adjust the hydrophobicity of the polymer matrix forming the microparticles;
selecting the L/G ratio of the polylactic-co-glycolic acid (PLGA) units to provide a sustained release of the active agent from the microparticles;
selecting the molar ratio of the amounts of the first to second crosslinkable precursors to adjust the hydrophobicity of the polymer matrix forming the microparticles, thus varying crosslinking density and structure of the polymer matrix;
selecting the molar ratio of the amounts of the first to second crosslinkable precursors to provide a sustained release of the active agent from the microparticles;
selecting the amount and/or particle size of the biodegradable microparticles to be included in the in the hydrogel, organogel xerogel;
adding a third crosslinkable precursor that is less hydrolysable than the first and second, optionally varying the molar ratios of the first, second and/or third precursors upon forming the biodegradable microparticles;
dispersing an active agent that has high water solubility in particulate form into the oil, using an organogel for forming the biodegradable microparticles.

In certain embodiments of the present invention, the release of the active agent is mainly controlled by diffusion of the active agent from the crosslinked polymer or from the hydrophobic liquid (e.g., oil) in the organogel forming the polymer matrix. The degradation rate of the polymer network offers another, independent additional mechanism for release control. In certain embodiments, the oil delays or accelerates the degradation, which can be used as another method of control the release of the active agent. When the active agent dispersed in the oil is released from the organogel microparticles together with the oil, the release rate of the agent will be essentially influenced or determined by the diffusion rate of the oil into the surrounding tissue or bodily environment. In other embodiments, the active agent may diffuse out of the oil more readily than the oil out of the polymer network.

When in contact with aqueous body fluids, the biodegradable microparticles of the present invention swell by taking up water. The degree of swelling is largely determined by the gel-forming components used and their hydrophobicity/hydrophilicity. Swelling may result in an increase of length and/or diameter dimensions of the organogels according to the invention of up to about 2000%, up to about 1000%, up to about 100%, up to about 95%, up to about 90%, up to about 80%, up to about 75%, up to about 70%, up to about 60%, up to about 50%, up to about 40%, up to about 30%, up to about 20% or up to about 10%. In certain embodiments, swelling may result in an increase of length and/or diameter dimensions of the organogels of at least a ratio of 1.05, 1.1, 1.2, 1.5 or 2 and can be a range with any of the above values.

However, even after swelling, in certain embodiments, the biodegradable microparticles including drug-delivery system of the present invention retains its shape or substantial shape over extended periods of time due to crosslinking of the polymer components. In certain embodiments, the polymer network of the drug delivery system and/or the microparticles will only substantially degrade after all active agent has been released, or at least after most of the active agent, for example at least about 50 wt.-%, at least about 60 wt.-%, at least about 70 wt.-%, at least about 80 wt.-%, at least about 90 wt.-% or at least about 99 wt.-%, or at least about 100 wt.-% of the active agent has been released.

For example, water replacing the oil in organogel microparticles over time may steadily dissolve hydrophilic active agents dispersed but not dissolved in the oil, which can be used to further control the active agent release. In this embodiment, the active agent release is mainly or completely controlled diffusion of the active agent through the oil and polymer into the surrounding tissue. In certain embodiments, another factor influencing or determining the release of the active agent dispersed in the oil when the active agent release rate is largely independent of the diffusion rate of the hydrophobic liquid, for example when it is an hydrophilic agent dispersed as solid particles in the oil, is the rate of diffusion of water into the organogel particles and/or the oil, thereby subsequently dissolving and eluting the active agent from the organogel into the surrounding aqueous environment.

Furthermore, in certain embodiments, water slowly replacing the oil slowly transforms the organogel microparticles into hydrogel microparticles, which are, however still crosslinked so maintaining their shape, but may then be easier (bio-) degraded by hydrolysis and/or enzymatic reactions after the microparticles or the drug-delivery system is depleted of active agent and/or oil.

The overall release of the active agent is controlled by at least one, or by a combination of all these release mechanisms.

In the embodiments of the present invention involving PLGA units in the crosslinked polymer or organogel matrix, still another mechanism can be utilized to influence or control the release of the active agent. The hydrophobic properties of the covalently crosslinked polymeric network in the body of a patient may be varied by adjusting the ratio of lactic acid to glycolic acid units. By changing or selecting the L/G ratio of the polylactic-co-glycolic acid (PLGA) units, the hydrophobicity of the polymeric network can be altered. More hydrophobic lactic acid (L) units will increase the hydrophobicity of the polymer, and reduce swelling and water uptake; increasing the content of relatively more hydrophilic glycolic acid (G) units will decrease the hydrophobicity of the polymer, and will increase swelling and water uptake of the polymer.

Another possibility to adjust the hydrophobicity of the polymer network is offered in embodiments of the invention by varying and/or selecting the molar ratio of the first to second crosslinkable precursors. Using hydrophobic precursors in a higher amount combined with more hydrophilic precursors such as PEG units, and vice versa, allow adjusting the swelling and hydrophobic liquid and/or active agent release.

Adding a third crosslinkable precursor that is different in its hydrophobicity than the first and second precursors and varying the molar ratios of the components can be further used to influence the swelling and hydrophobic liquid and/or active agent release, and the diffusion rates of active agent, hydrophobic liquid and/or water.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Materials and Abbreviations Used in the Examples

4a20kPLGA-NHS is a four arm 20 kDalton electrophile functionalized polymeric precursor obtained by functionalization of commercially available 4a20kPLGA (having an L/G ratio of 50:50) with N-hydroxy succinimide (NHS).

TAEA is tris(2-aminoethyl)amine, which is commercially available from Sigma-Aldrich/Merck. DMC is dimethyl carbonate.

DCM is dichloromethane.

PBS is phosphate buffered saline of physiological salt concentration, pH7.4.

1% aqueous PVA solution was obtained by diluting a 4% aqueous PVA solution, commercially available from Sigma-Aldrich.

### Example 1

### Heat Stability of Crosslinked PLGA Microspheres

Biodegradable microparticles comprising a crosslinked PLGA polymer matrix but no active agent were produced (sample No. RH-558-1) using 4a20k-PLGA-NHS as an electrophile functionalized polymeric precursor, TAEA as the small molecule nucleophile crosslinker and DCM as the solvent.

4a20k-PLGA-NHS (1000 mg) were dissolved in DCM (2 mL) and a part of the solution the solution filled into a first syringe. Separately, TAEA (10 mg) was mixed into in DCM (2 mL) and the solution filled into a second syringe. A Fibrijet® Y type mixer (FIG. 2) was used to continuously merge the two solutions and to inject the mixed precursor solutions through a 21G needle into an agitated (angled impeller at 700 rpm) 1% aqueous PVA solution (300 mL). The obtained emulsion was stirred overnight, DCM was extracted and evaporated, and the microparticles hardened. Thereafter, the formed microparticles were removed from the solution and washed with water. The washed microparticles were then sieved into two fractions the microparticles into particles having a diameter of >106 μm and particles having a diameter of 20-106 μm. The remaining water was removed from the microparticles by lyophilisation to yield dry, crosslinked PLGA microparticles.

TABLE 1
Composition details of the inventive example.

Example 1 (RH558-1)

| | | Amount | % w/w |
|---|---|---|---|
| Ingredients | | (mg) | dry |
| Electrophile | 4a20k-PLGA- | 1000 | 99 |
| | NHS | | |
| Nucleophile / | TAEA | 10 | |
| Crosslinker | | | |
| Active agent | — | — | — |
| Solvent | DCM | | |
| Total | | 1010 | |

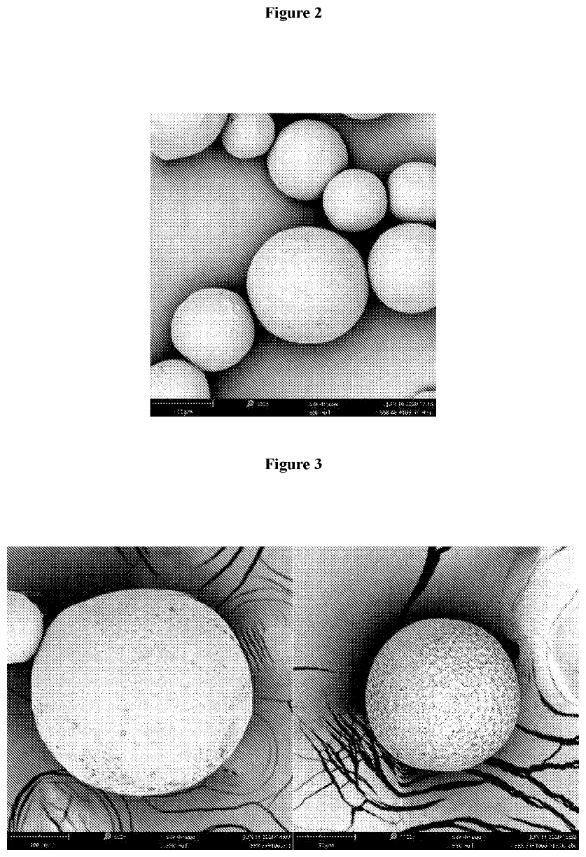

The microparticles having a diameter of >106 μm obtained according to Example 1 were heated on a glass slide at 80° C. for 2 hours. The SEM image of FIG. 3 shows that the microparticles conserved their shape, indicating that no melting of the microparticles occurred and that the microparticles are heat stable.

### Example 2

Biodegradable microparticles comprising an organogel and an active agent were produced (sample No. RH-558-7) using 4a20k-PLGA-NHS as an electrophile functionalized polymeric precursor, travoprost as the active agent, TAEA as the small molecule nucleophile/crosslinker and DCM as the solvent.

4a20k-PLGA-NHS (500 mg) and travoprost (500 mg) were dissolved in DCM (2.5 mL) and the solution filled into a first syringe. Separately, TAEA (5 mg) was dissolved in DCM (2.5 mL) and the solution filled into a second syringe. Like in Example 1, a Fibrijet® Y type mixer (FIG. 2) was used to continuously merge the two solutions and to inject the mixed solution through a 21G needle into an agitated (angled impeller at 700 rpm) 1% aqueous PVA solution (300 mL). The obtained emulsion was stirred overnight to extract and evaporate DCM and harden the microparticles. Thereafter, the formed microparticles were removed from the solution and washed with water. The washed microparticles were then sieved into two fractions the microparticles into particles having a diameter of >106 μm and particles having a diameter of 20-106 μm. The remaining water was removed from the microparticles by lyophilisation to yield dry, travoprost-loaded crosslinked PLGA microparticles. Travoprost content was determined by the following method: Travoprost in the microparticles was extracted with acetonitrile and diluted with PBS. The extract was analysed by UPLC on a Water Acquity system (Waters Corporation, U.S.) with a Acquity BEH C18, 2.1 mm×50 mm, 1.7 μm particle column. The mobile phase was isopropanol and 50:50 0.1% TFA:Acetonitrile with a gradient flow. The run time was 5 min with travoprost peak at 1.4 minute and UV detection at 220 nm. The results are summarized in Table 2 below.

TABLE 2
Composition details of the inventive example.

Example 1 (RH558-7)

| | | Amount | % w/w |
|---|---|---|---|
| Ingredients | | (mg) | dry |
| Electrophile | 4a20k-PLGA- | 500 | 50 |
| | NHS | | |
| Nucleophile / | TAEA | 5 | |
| Crosslinker | | | |
| Active agent | Travoprost | 500 | 50 |
| Solvent | DCM | | |
| Total | | 1005 | |

TABLE 3
Travoprost loading of the inventive
crosslinked PLGA microparticles.

| | Average Travoprost loading |
|---|---|
| Inventive Example | [wt %] |
| MP diameter 20-106 μm | 41.8 |
| MP diameter >106 μm | 46.9 |

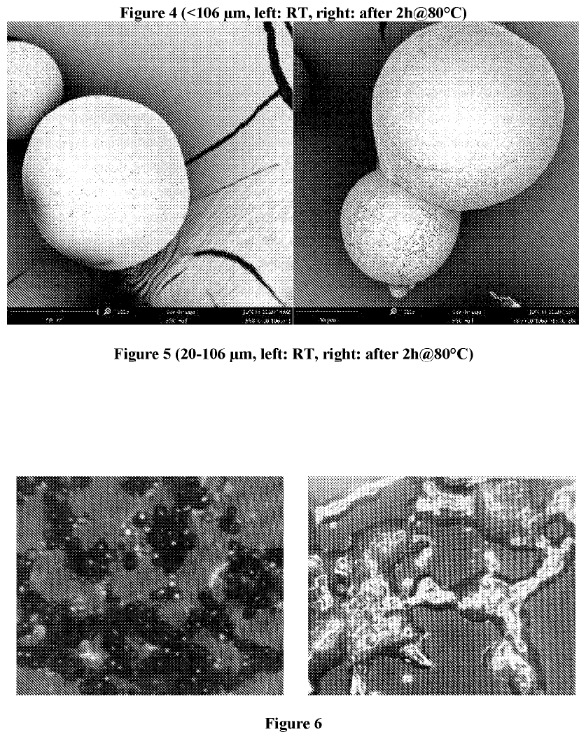

The microparticles obtained according to Example 1, and comparative microparticles made of non-crosslinked PLGA according to prior art methods were each heated on a glass slide at 80° C. for 2 hours. The SEM images of FIGS. 4 and 5 show, that the microparticles having a diameter of 20-106 μm (FIG. 4) as well as the microparticles having a diameter of >106 μm (FIG. 5) mainly conserved their shape, indicating that no melting of the microparticles occurred and that the microparticles are heat stable. In FIGS. 4 and 5, the left picture is a taken at room temperature before heating, and the right picture is after heat treatment at 80° C. for 2 hours

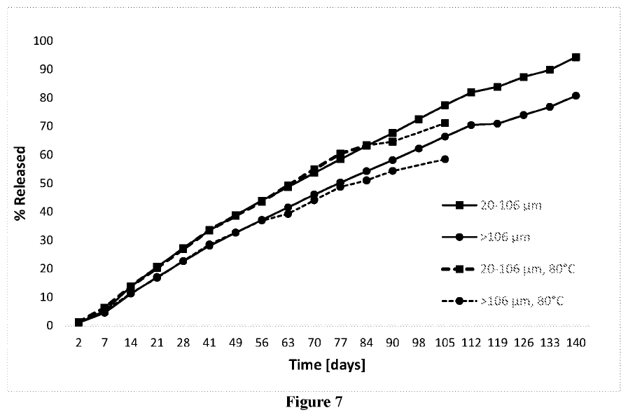

FIG. 6 is an image of the microparticles of Example 2 (left side, colored with violet dye (D&C Violet #2) for better visualization) and comparative, non-crosslinked PLA microparticles (right side) prepared as described in Example 1 of US2018/0085307A1, after the heat treatment at 80° C. for 2 h. While the heat stable microparticles of present Example 2 conserved their shape, the comparative PLGA microparticles show a collapsed shape due to melting.

### Example 3

### Heat Treatment and Particle Diameter Influence on In-Vitro Release of Active Agent

Crosslinked microparticles as prepared in present Example 2 with the two particle size fractions described there (20-106 μm and >106 μm) without heat treatment and after a heat treatment at 80° C. for 2 h were subjected to accelerated in-vitro travoprost release kinetics measurements at 40° C. in 50 mL of modified 1×PBS buffer (including 0.5% PEG40 Castor oil and 0.01% NaF), so that the 100% release corresponds to 5 to 10 times below sink conditions. The in-vitro release data is shown in FIG. 7.

As can be seen in FIG. 7, the active agent (travoprost) is released in a constant, slow, sustained manner following substantially zero order kinetics over an extended period of time and no burst is seen initially. With the polymeric microparticles expected to degrade substantially only after 6 months, the release kinetics of the active agent is diffusion controlled. Effects of degradation of the microparticles are not seen.

Further, the smaller microparticles having a diameter size of 20-106 μm release the travoprost faster than microparticles having a diameter of >106 μm. This can be explained by the bigger surface-area to volume ratio of microparticles having a diameter of 20-106 μm compared to microparticles having a diameter size of >106 μm, which leads to higher diffusion rates, since diffusion takes place at the surface of the particles.

FIG. 7 also shows that the active agent release of the crosslinked PLGA microparticles is essentially unaffected by the heat treatment at 80° C. for 2 hours for substantial period of time, as the release is similar to the corresponding unheated microparticles.

### Example 4

Comparison with Conventional Microparticles

The active agent release from crosslinked microparticles as prepared in present Example 2 from the particle size fraction of >106 μm has been compared with non-crosslinked PLA microparticles, prepared in the same way as described in paragraph [0149] of US2018/0085307A1 or [0501] of US2021/0251893A1, designated 4.5A PLA, 8A PLA and 10.5A PLA (A designates acid capped linear PLA, and the polymers differ in molecular weight: 4.5A has an inherent viscosity of 0.3-0.4 dL/g, 8A has an inherent viscosity of 0.7-0.9 dL/g and 10.5A has an inherent viscosity of 1.0-1.1 dL/g, (all measured in chloroform at 0.5% w/v, a temperature of 30° C., Ubbelohde Viscosimeter size 0 B, an approximate viscosimeter constant (C) of 0.005 mm²/s², a capillary working length (L) of 40 mm, a bulb volume (V) of 3.0 mL, a capillary inside diameter (d) of 0.46 mm, and an approximate flow time of solvent (CHCl₃) of 78 s).

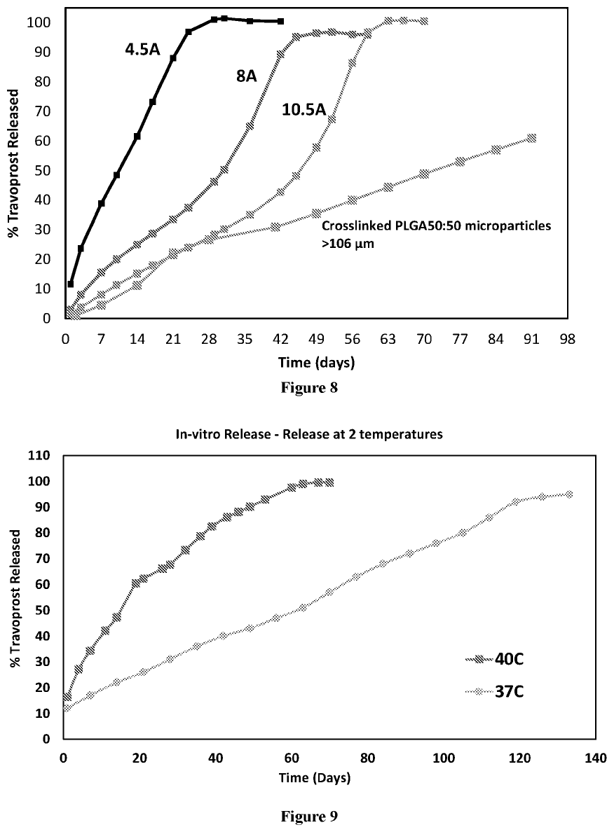

All microparticles were subjected to accelerated in-vitro travoprost release kinetics measurements at 40° C. in 50 mL of modified 1×PBS buffer as in Example 3, so that the 100% release corresponds to 5 to 10 times below sink conditions. The in-vitro release data is shown in FIG. 8.

As can be seen in FIG. 8, the release of travoprost from the non-crosslinked PLA microparticles is significantly faster and less linear, with a large burst of the 4.5A PLA particles, compared to the substantially zero order release from the crosslinked PLGA microparticles of Example 2 that do not show any initial burst.

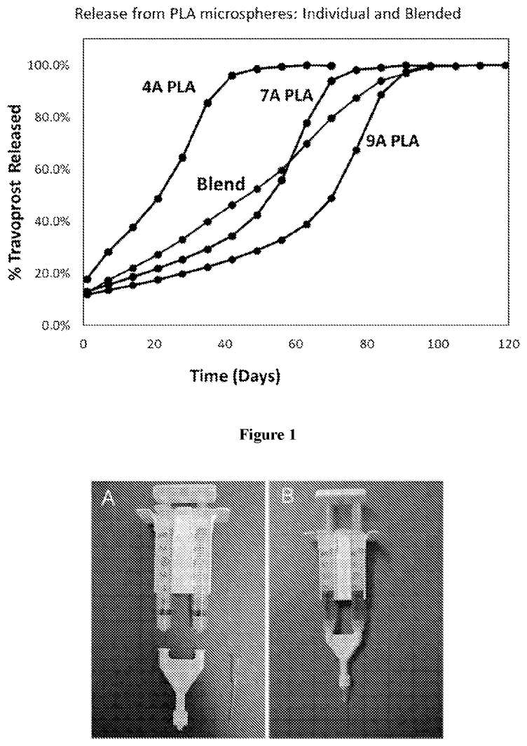

As a further comparison, FIG. 9 shows the in-vitro release of travoprost from a blend of different non-crosslinked PLA microparticles at 37° C. and 40° C. in modified 1×PBS buffer as described in Example 3. A clear initial burst of the microparticles releasing >10% of the travoprost can be seen at both temperatures. Further, while the release of travoprost from the polymer blend microparticles (similar to the blend curve in FIG. 1) at 37° C. is almost constant, this release from blended non-crosslinked PLA microparticles is not temperature stable, and a non-linear release at 40° C. is observed. When compared with the linear, zero order release of the crosslinked PLGA microparticles of the present invention at the same temperature (see FIG. 8), this demonstrates better control of the release kinetics by using crosslinked microparticle drug delivery systems of embodiments of the present invention.

The invention is further described by the following lists of items.

### FIRST LIST OF ITEMS

1. Biodegradable microparticles for sustained release drug-delivery, comprising an active agent and a covalently and three-dimensionally crosslinked biodegradable polymer matrix, wherein the crosslinked biodegradable polymer includes polymer units of at least one of crosslinked polylactic acid (PLA), polyglycolic acid (PGA), polylactic-co-glycolic acid (PLGA), poly(vinylpyrrolidone), poly-p-dioxanone, poly(trimethylene carbonate), polycaprolactone, and/or polyvinyl alcohol, mixtures or copolymers of any of these.
2. The biodegradable microparticles of item 1, wherein the microparticles are microspheres having a substantially spherical shape.
3. The biodegradable microparticles of item 1 or 2, wherein the active agent is dispersed, embedded or encapsulated in the crosslinked polymer.
4. The biodegradable microparticles of any one of the preceding items, wherein the crosslinked polymer further comprises at least one oil, and the microparticles consisting of an organogel comprising at least one active agent and the at least one oil in the crosslinked polymer.
5. The biodegradable microparticles of any one of the preceding items, wherein the polymer units further include units selected from at least one of polyethylene glycol (PEG), polypropyleneglycol (PPG), and/or at least one of polyaminoacids, glycosaminoglycans, polysaccharides, or proteins and optionally copolymers or mixtures thereof with any of the polymer units of item 1.
6. The biodegradable microparticles of any one of item 5, wherein the biodegradable, covalently and three-dimensionally crosslinked polymer comprises a plurality of hydrophobic polymer units and/or hydrophilic polymer units.
7. The biodegradable microparticles of item 6, wherein the hydrophobic polymer units are selected from at least one of polylactic acid (PLA), and polylactic-co-glycolic acid (PLGA) units, and the hydrophilic polymer units are selected from at least one of polyethylene glycol units, polypropylene glycol units, or polyglycolic acid (PGA), preferably polyethylene glycol units.
8. The biodegradable microparticles of any one of the preceding items, wherein the covalently and three-dimensionally crosslinked biodegradable polymer comprises or consists of crosslinked polylactic-co-glycolic acid (PLGA) units.
9. The biodegradable microparticles of item 8, wherein the polylactic-co-glycolic acid (PLGA) units have an L/G ratio (in % L or G units) ranging from 0:100 to 100:0, or 1:99 to 99:1, or 10:90 to 90:10, or 25:75 to 75:25, preferably 50:50.
10. The biodegradable microparticles of any one of the preceding items, wherein each of the polymer units having an average molecular weight (Mw) in the range from about 1,000 to about 100,000 Daltons, or from about 10,000 to about 60,000 Daltons, or from about 15,000 to about 50,000 Daltons.
11. The biodegradable microparticles of any one of the preceding items, wherein the polymer is covalently crosslinked by linkages between polymer units.
12. The biodegradable microparticles of item 11, wherein the linkages are selected from the group consisting of amine, amide, urethane, ester, anhydride, ether, acetal, ketal, nitrile, isonitrile, isothiocyanate, isourea, hydrazone, oxime, or imine bonds, optionally linkages produced by polycondensation, radical polymerization or click chemistry reactions, and combinations thereof.
13. The biodegradable microparticles of any one of the preceding items, wherein the active agent is selected from at least one of a therapeutically active agent or a diagnostically active agent, or combinations thereof.
14. The biodegradable microparticles of any one of the preceding items, wherein the therapeutically active agent is selected from steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, viruses, viruses for gene delivery such as AAV, etc., or any combinations thereof.
15. The biodegradable microparticles of any one of the preceding items, wherein the microparticles have a particle size (diameter) of 0.1-1000 μm, or 1 to 150 μm, 1 to 100 μm, 20 to 75 μm, 10 to 106 μm or 20 to 55 μm, determined by sieving, or have an average diameter ranging from 0.1-1000 μm, or 1 to 150 μm, 1 to 100 μm, 20 to 75 μm, 10 to 106 μm or 20 to 55 μm, determined by laser diffraction.
16. The biodegradable microparticles of any one of the preceding items, wherein the microparticles have a particle size distribution such as a D50 particle size of less than about 100 μm, or less than about 50 μm, or less than about 20 μm and/or a D90 particle size of less than about 200 μm, or less than about 50 μm, or a D90 particle size of about 100 μm or less, or 30 μm or less and/or a D90 particle size of about 20 μm or less, determined by laser diffraction.
17. The biodegradable microparticles of any one of the previous items, consisting of a blend of microparticles having different particle sizes and/or having a different polymer matrix, and/or including different active agents.
18. The biodegradable microparticles of any one of the preceding items, wherein the crosslinked biodegradable polymer has a glass transition temperature below human body temperature, such as below 37° C., or below 36° C., below 30° C., below 25° C., below 20° C., or below 10° C., and/or wherein the polymer has a melt temperature above 40° C., 45° C., 50° C., 60° C. or 70° C.
19. The biodegradable microparticles of any one of the preceding items, providing for a release of a therapeutically or diagnostically effective amount of the active agent for a period of time, such as up to 1 year, up to 9 months, up to 6 months, op to 3 months, up to 1 month, or up to about 25 days after administration, preferably up to about 14 days, or up to about 21 days after administration, wherein optionally the active agent release is substantially constant in a temperature range of 30° C. to 45° C., or 36 to 43° C.

### SECOND LIST OF ITEMS

1. Biodegradable microparticles for sustained release drug-delivery, comprising an organogel comprising at least one active agent, at least one oil, and a covalently and three-dimensionally crosslinked biodegradable polymer matrix, wherein the crosslinked biodegradable polymer includes polymer units of at least one of polyethylene glycol (PEG), polypropyleneglycol (PPG), polyvinyl alcohol, poly(vinylpyrrolidone), polylactic acid (PLA), polyglycolic acid (PGA), polylactic-co-glycolic acid (PLGA), poly-p-dioxanone, poly(trimethylene carbonate), polycaprolactone, and/or polyvinyl alcohol, random or block copolymers or combinations or mixtures of any of these, or one or more of polyaminoacids, glycosaminoglycans, polysaccharides, or proteins.
2. The biodegradable microparticles of item 1, wherein the microparticles are microspheres having a substantially spherical shape.
3. The biodegradable microparticles of item 1 or 2, wherein the active agent is dispersed, embedded or encapsulated in the crosslinked polymer.
4. The biodegradable microparticles of any one of the preceding items, wherein the at least one oil is a liquid at human body temperature, such as a temperature of about 37° C. or lower, or liquid in the range of 0° C. to 40° C., or 10° C. to 38° C., or 15° C. to 37° C., or 25° C. to 37° C., or at 37° C.
5. The biodegradable microparticles of any one of the preceding items, wherein the at least one oil is selected from the group comprising triethyl citrate, acetyl triethyl citrate (ATEC), acetyl tributyl citrate (ATBC), α-tocopherol (vitamin E), α-tocopherol acetate; plant or vegetable oils such as sesame oil, olive oil, soybean oil, sunflower oil, coconut oil, canola oil, rapeseed oil, nut oils such as hazelnut, walnut, pecan, almond, cottonseed oil, corn oil, safflower oil, linseed oil, etc., ethyl oleate, castor oil and derivatives thereof (Cremophor®), lipids being liquid at 37° C. or lower, such as saturated or unsaturated fatty acids, monoglycerides, diglycerides, triglycerides (Myglyols®), phospholipids, glycerophospholipids, sphingolipids, sterols, prenols, polyketides, hydrophobic biodegradable liquid polymers, low melting point waxes such as plant, animal or synthetic waxes, lanolin, jojoba oil, or combinations thereof.
6. The biodegradable microparticles of any one of the preceding items, wherein the biodegradable, covalently and three-dimensionally crosslinked polymer comprises a plurality of hydrophobic polymer units and/or hydrophilic polymer units.
7. The biodegradable microparticles of item 6, wherein the hydrophobic polymer units are selected from at least one of polylactic acid (PLA), and polylactic-co-glycolic acid (PLGA) units, and the hydrophilic polymer units are selected from at least one of polyethylene glycol units, polypropylene glycol units, or polyglycolic acid (PGA), preferably polyethylene glycol units.
8. The biodegradable microparticles of any one of the preceding items, wherein the covalently and three-dimensionally crosslinked biodegradable polymer comprises or consists of crosslinked polylactic-co-glycolic acid (PLGA) units.
9. The biodegradable microparticles of item 8, wherein the polylactic-co-glycolic acid (PLGA) units have an L/G ratio (in % L or G units) ranging from 0:100 to 100:0, or 1:99 to 99:1, or 10:90 to 90:10, or 25:75 to 75:25, preferably 50:50.
10. The biodegradable microparticles of any one of the preceding items, wherein each of the polymer units having an average molecular weight (Mw) in the range from about 1,000 to about 100,000 Daltons, or from about 10,000 to about 60,000 Daltons, or from about 15,000 to about 50,000 Daltons.
11. The biodegradable microparticles of any one of the preceding items, wherein the polymer is covalently crosslinked by linkages between polymer units.
12. The biodegradable microparticles of item 11, wherein the linkages are selected from the group consisting of amine, amide, urethane, ester, anhydride, ether, acetal, ketal, nitrile, isonitrile, isothiocyanate, isourea, hydrazone, oxime, or imine bonds, optionally linkages produced by polycondensation, radical polymerization or click chemistry reactions, and combinations thereof.
13. The biodegradable microparticles of any one of the preceding items, wherein the active agent is selected from at least one of a therapeutically active agent or a diagnostically active agent, or combinations thereof.
14. The biodegradable microparticles of any one of the preceding items, wherein the therapeutically active agent is selected from steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, viruses, viruses for gene delivery such as AAV, etc., or any combinations thereof.
15. The biodegradable microparticles of any one of the preceding items, wherein the microparticles have a particle size (diameter) of 0.1-1000 μm, or 1 to 150 μm, 1 to 100 μm, 20 to 75 μm, 10 to 106 μm or 20 to 55 μm, determined by sieving, or have an average diameter ranging from 0.1-1000 μm, or 1 to 150 μm, 1 to 100 μm, 20 to 75 μm, 10 to 106 μm or 20 to 55 μm, determined by laser diffraction.
16. The biodegradable microparticles of any one of the preceding items, wherein the microparticles have a particle size distribution such as a D50 particle size of less than about 100 μm, or less than about 50 μm, or less than about 20 μm and/or a D90 particle size of less than about 200 μm, or less than about 50 μm, or a D90 particle size of about 100 μm or less, or 30 μm or less and/or a D90 particle size of about 20 μm or less, determined by laser diffraction.
17. The biodegradable microparticles of any one of the previous items, consisting of a blend of microparticles having different particle sizes and/or having a different polymer matrix, and/or including different active agents.
18. The biodegradable microparticles of any one of the preceding items, wherein the crosslinked biodegradable polymer or organogel has a glass transition temperature below human body temperature, such as below 37° C., or below 36° C., below 30° C., below 25° C., below 20° C., or below 10° C., and/or wherein the polymer has a melt temperature above 40° C., 45° C., 50° C., 60° C. or 70° C. 19. The biodegradable microparticles of any one of the preceding items, providing for a release of a therapeutically or diagnostically effective amount of the active agent for a period of time, such as up to 1 year, up to 9 months, up to 6 months, op to 3 months, up to 1 month, or up to about 25 days after administration, preferably up to about 14 days, or up to about 21 days after administration, wherein optionally the active agent release is substantially constant in a temperature range of 30° C. to 45° C., or 36 to 43° C.

### THIRD LIST OF ITEMS

1. Biodegradable microparticles for sustained release drug-delivery, comprising an active agent and a covalently and three-dimensionally crosslinked biodegradable polymer matrix, wherein at least one of the following conditions is fulfilled:
   the crosslinked biodegradable polymer includes at least one of crosslinked polylactic acid (PLA), polyglycolic acid (PGA), polylactic-co-glycolic acid (PLGA), poly(vinylpyrrolidone), poly-p-dioxanone, poly(trimethylene carbonate), polycaprolactone, and/or polyvinyl alcohol, or copolymers of any of these;
   the biodegradable microparticles include an organogel comprising the covalently crosslinked biodegradable polymer and an oil.
2. The biodegradable microparticles of item 1, wherein the microparticles are microspheres having a substantially spherical shape.
3. The biodegradable microparticles of item 1 or 2, wherein the active agent is dispersed, embedded or encapsulated in the organogel.
4. The biodegradable microparticles of any one of the preceding items, wherein the organogel is formed by chemically crosslinking at least one multifunctional precursor to form the covalently and three dimensionally crosslinked polymer matrix, optionally in the presence of an oil.
5. The biodegradable microparticles of item 4, wherein the at least one precursor has a functionality for chemical crosslinking of greater than 2, such as 3 to 10, or 3 to 9, or 4 to 8, or 4.
6. The biodegradable microparticles of any one of items 4 or 5, wherein the at least one precursor is a dendrimer or multi-arm precursor having a core and from 2 to 10 arms, or 3 to 10 arms, 4 to 8 arms, or 4 or 6 arms, each arm comprising a polymer unit and having a terminus.
7. The biodegradable microparticles of any one of items 4 to 6, wherein the polymer units are selected from polyethylene glycol (PEG), polypropyleneglycol (PPG), polyvinyl alcohol, poly(vinylpyrrolidone), polylactic acid (PLA), polyglycolic acid (PGA), polylactic-co-glycolic acid (PLGA), poly-p-dioxanone, poly(trimethylene carbonate), polycaprolactone, random or block copolymers or combinations or mixtures of any of these, or one or more units of polyaminoacids, glycosaminoglycans, polysaccharides, or proteins.
8. The biodegradable microparticles of any one of items 4 to 7, wherein the polymer units in the arms of the multi-arm precursors are the same or different types of polymer units.
9. The biodegradable microparticles of any one of items 4 to 8, further including at least one crosslinker having at least two functional groups, or more than two functional groups, preferably a small molecule amine such as tris(2-aminoethyl)amine (TAEA) or trilysine.
10. The biodegradable microparticles of any one of items 4 to 8, wherein the organogel comprises at least two crosslinkable dendrimer or multi-arm precursors crosslinked with each other.
11. The biodegradable microparticles of any one of items 4 to 10, wherein the dendrimer or multi-arm precursor(s) comprises a functional group on at least 3 of its arm termini, or on each terminus.
12. The biodegradable microparticles of any one of items 4 to 11, wherein the polymer matrix is formed from a first multi-arm precursor comprising a first functional group, and a second multi-arm precursor or crosslinker comprising a second functional group, the functional groups being located at the terminus of the arms of the precursor or crosslinker, wherein the first or second functional group may be directly grafted to the precursor terminus, or via a linker molecule.
13. The biodegradable microparticles of item 12, wherein each of the first functional group and the second functional group is selected from an electrophile and a nucleophile, functional groups for click chemistry, functional groups for cycloadditions, particularly 1,3 dipolar cycloadditions, hetero-Diels-Alder cycloadditions, functional groups for nucleophilic ring openings, functional groups for non-aldol type carbonyl reactions, functional groups for addition reactions to carbon-carbon multiple bonds, polymerizable vinyl groups, or combinations thereof.
14. The biodegradable microparticles of item 13, wherein each of the first functional group and the second functional group is selected from an electrophile and a nucleophile, and the reaction between the first functional group and second functional group is an electrophile-nucleophile reaction that forms a covalent bond.
15. The biodegradable microparticles of item 14, wherein the nucleophile is selected from one of an amine, such as a primary amine, a hydroxyl, an alcohol, a thiol, an azide anion, and a carboxyl group.
16. The biodegradable microparticles of item 14 or 15, wherein the electrophile is selected from activated ester groups such as succinimidyl esters, succinimidyl carbonates; nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halogen.
17. The biodegradable microparticles of any one of items 14 to 16, wherein the nucleophile is an amine group, particularly a primary amine, and the electrophile is an activated ester group, particularly a succinimidyl ester selected from succinimidyl succinate, succinimidyl glutarate, succinimidyl adipate, succinimidyl azelate, or succinimidyl glutaramide.
18. The biodegradable microparticles of item 13, wherein each of the first functional group and the second functional group are selected from functional groups for click chemistry, particularly functional groups for cycloadditions, particularly 1,3 dipolar cycloadditions, [3+2] cycloadditions such as alkene-nitrone cycloadditions or alkyne-nitrone cycloadditions, [4+2] cycloadditions, hetero-Diels-Alder cycloadditions; functional groups for thiol-ene reactions; functional groups for nucleophilic ring openings; functional groups for non-aldol type carbonyl reactions; functional groups for addition reactions to carbon-carbon multiple bonds; functional groups for Michael-type additions.
19. The biodegradable microparticles of item 18, wherein the first functional group is an alkyne compound such as a dibenzocyclooctyne (DBCO), or a bicyclo[6.1.0]-nonyne (BCN); or a norbornene, or a trans-cyclooctene (TCO), and the second functional group is an azide, a 3,4 dihydroxyphenylacetic acid (DHPA), or a tetrazine (Tz).
20. The biodegradable microparticles of item 19, wherein the DBCO, BCN, norbornene, TCO, azide, DHPA and Tz functional groups are grafted to the termini of the multi-arm precursor via a linker such as an acid group, a diacid group, a functionalized aliphatic, heteroaliphatic, or aromatic or heteroaromatic group.
21. The biodegradable microparticles of item 13, wherein the first and second functional groups are selected for a [3+2] cycloaddition reaction such as alkene-nitrone cycloadditions or alkyne-nitrone cycloadditions, or wherein the first and second functional group are selected for a [4+2] cycloaddition reaction, particularly a hetero Diels-Alder reaction, wherein the first functional group is an aldehyde or imine compound, and the second functional group is a 1,3 diene compound, an unsaturated carbonyl compound, or a nitroso-alkene compound.
22. The biodegradable microparticles of item 13, wherein the first and second functional group are selected for a thiol-ene reaction, wherein the first functional group is a thiol compound and the second functional group is an alkene, preferably a terminal alkene, or wherein the first and second functional group are selected for nucleophilic ring openings, wherein the first functional group is selected from an epoxide, thiirane, aziridine, or lactam, and the second functional group is nucleophile.
23. The biodegradable microparticles of item 13, wherein the first and second functional group are selected for non-aldol type carbonyl reactions, wherein the first functional group is an aldehyde or ketone compound, and the second functional group is a primary amine, a hydrazide, acyl hydrazide or aminooxy compound, to form an imine, amide, isourea, hydrazone, acyl hydrazone or oxime linkage.
24. The biodegradable microparticles of item 13, wherein each of the first functional group and the second functional group are selected from polymerizable vinyl groups and acrylates such as (meth)acrylic acid, (meth)acrylic acid esters, acrylamides, fumaric acid, maleic acid and combinations thereof, wherein crosslinking is induced thermally or photochemically, optionally with the use of initiators such as photo initiators such as free radical photo initiators (Norish I type such as 2,2-dimethoxy-1,2-diphenyl-ethan-1-one, 2-Hydroxy-2-methyl-1-phenylpropanone, 1-hydroxy-cyclohexylphenylketone; or Norish II type such as benzophenone and its derivative and isopropyl thioxanthone in combination with a synergist such as tertiary amines2-ethylhexyl-(4-N,N-dimethyl amino)benzoate, and 2-ethyl-(4-N,N-dimethylamino)benzoate); or cationic photo-initiators.

### FOURTH LIST OF ITEMS

1. A method for manufacturing the biodegradable microparticles for sustained release drug-delivery according to any one of the first to third list of items, the method being selected from one of the following techniques: emulsion solvent evaporation-extraction, emulsion solvent diffusion, supercritical fluid emulsion, coacervation, spray drying, hydrogel template, microfluidic systems, membrane extrusion emulsification, particle replication in non-wetting templates (PRINT) technique, electro hydrodynamic atomization (EHDA) or electro-spraying, or particles obtained from gas saturated solutions (PGSS) method, or by 3D printing.
2. A method according to item 1, the method comprising the steps of:
   (1) forming a gel comprising a covalently crosslinked polymer in the presence of at least one active agent, optionally at least one oil and optionally a first solvent,
   (2) producing microparticles wherein the at least one active agent is dispersed within the covalently crosslinked polymer, and
   (3) optionally removing solvent.
3. The method of item 2, comprising the steps of:
   (a) dissolving at least one of the polymer precursors in a first solvent, producing a first mixture;
   (b) providing a second mixture comprising a crosslinker in a second solvent;
   (c) adding at least one active agent, and optionally an oil, to at least one of the first mixture or second mixture;
   (d) combining the first mixture and the second mixture to produce a first phase;
   (e) providing a second phase comprising a third solvent, that is immiscible with the first and second solvents;
   (f) introducing, under agitation, the first phase into the second phase, thereby producing an emulsion of dispersed first phase in the second phase; and
   (g) removing the first, second and/or third solvent.
4. The method of item 2 or 3, wherein the step of producing microparticles (step (2)) or the step f) comprises forcing the first phase through a mesh or injecting the first phase into the agitated second phase, the first and/or second solvent and/or third solvent optionally comprising additives such as emulsifiers, surfactants, dispersing adjuvants, or porogens, to form microspheric or nanospheric particles.
5. The method of any one of items 2 to 4, wherein the first solvent and/or second solvent is an organic solvent wherein the precursors are soluble, and the third solvent is a solvent wherein the first phase and/or the formed organogel thereof is insoluble.
6. The method of item 5, wherein the first or second solvent is selected from acetone, acetonitrile, benzyl alcohol, chloroform, dichloromethane (DCM), dioxane, dimethyl carbonate, DMSO, ethanol, ethyl acetate, ethyl formate, ethyl propionate, glycofurol, hexafluoro-isopropanol, isosorbide dimethyl ether, isopropanol, methyl chloride, methylene chloride, methyl ethyl ketone, N-methylpyrrolidone, propylene carbonate, or tetrahydrofuran, or any mixtures thereof, and the third solvent is water, an alcohol such as methanol, ethanol or propanol, or any mixture thereof.
7. The method of any one of items 4 to 6, wherein the additives are selected from surfactants or emulsifiers such as polyvinyl alcohol (PVA), polyethylene glycol sorbitan monolaurate (Tween®), sorbitan monolaurate (Span®), sodium dodecyl sulfate (SDS); and/or porogens such as inorganic salts (NaCl, KCl, sodium or potassium carbonates or bicarbonates, ammonium bicarbonate), Pluronics; sodium or potassium oleate; gelatin; mustard oil, mineral oil; cyclodextrins; carbohydrates, bovine serum albumin (BSA); photo initiators, radical polymerization initiators, and combinations thereof
8. The method of any one of items 2 to 7, wherein steps 1 and 2 make use of oil-in-water emulsion or water-in-oil emulsion technology, or combinations thereof, particularly single or double emulsion technique, or microfluidic technology, or combinations thereof.
9. The method of any one of items 2 to 8, wherein removal of the first and/or second and/or third solvent is done by one of hot air convection or direct drying, indirect or contact drying, spray drying, dielectric drying, vacuum drying, freeze drying, supercritical or superheated steam drying, or combinations of any of these.
10. A biodegradable microparticle for sustained drug-delivery, obtainable by a method on any one of the preceding items.

### FIFTH LIST OF ITEMS

1. A sustained release, biodegradable drug-delivery system, comprising the biodegradable microparticles for sustained release drug-delivery according to any one of the first to third lists of items above.
2. The drug-delivery system of item 1, wherein the biodegradable microparticles are incorporated into a hydrogel, xerogel or organogel.
3. The drug-delivery system of item lor 2, for coating a medical implant or for use as a medical implant.
4. A medical implant for sustained drug-delivery comprising the biodegradable microparticles for sustained release drug-delivery according to any one of the first to third lists of items above incorporated into a hydrogel, xerogel or organogel.
5. The drug-delivery system or implant of any one of items 1 to 4, wherein the system or implant is selected from the group consisting of an intraocular implant, intracaveal implant, intracameral implant, an implant for introduction into the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrascleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroid, tenon, cornea, cancer tissue, organ, prostate, breast, joint space, subdural, dental, subcutaneous, carpal tunnel, perivascular, surgically created space or injury, void space, and potential space.
6. The drug-delivery system or implant of any one of items 1 to 4, wherein the system or implant is obtained by extrusion or injection molding of a reaction mixture comprising the biodegradable microparticles dispersed in a hydrogel, xerogel or organogel or precursors thereof.
7. The drug-delivery system or implant of item 6, wherein the gelling occurs before and/or during extrusion or injection molding of the gel-forming mass comprising the biodegradable microparticles.
8. The drug-delivery system or implant of any one of items 2 to 7, wherein the content of the biodegradable microparticles embedded in the hydrogel, organogel or xerogel, with respect to the total weight of the drug-delivery system or implant, is about 10 wt.-% to about 35 wt.-%, or about 23 to about 27 wt.-%, or about 12 to about 17 wt.-%, or about 30 to about 35 wt.-%, or about 25 wt.-%, or about 15 wt.-%, or about 34 wt.-%. 9. The drug-delivery system or implant of any one of the preceding items, providing for a release of a therapeutically or diagnostically effective amount of the active agent for a period of time, such as up to 1 year, up to 9 months, up to 6 months, op to 3 months, up to 1 month, or up to about 25 days after administration, preferably up to about 14 days, or up to about 21 days after administration, wherein optionally the active agent release is substantially constant in a temperature range of 30° C. to 45° C., or 36 to 43° C.
10. A sustained release, biodegradable drug-delivery system or implant of any one of items 1 to 8, for use as a medicament.
11. A sustained release, biodegradable drug-delivery system or implant of any one of items 1 to 8, for use in treating a disease/medical condition of a patient, the use comprising the incorporation of the biodegradable microparticles according to any one of the first to third lists of items above into a carrier such as a hydrogel, organogel or xerogel, wherein the hydrogel, organogel or xerogel is formed in situ at a treatment site of the patient, or is prefabricated and delivered to or implanted at a treatment site of the patient in order to release the active agent from the microparticles over an extended period of time, or the carrier being a solvent or solvent system to produce an injectable suspension or dispersion.
12. A method for treating a disease/medical condition of a patient, the method comprising the incorporation of the biodegradable microparticles according to any one of the first to third lists of items above into a hydrogel, organogel or xerogel, wherein the hydrogel, organogel or xerogel is formed in situ at a treatment site of the patient, or is prefabricated and delivered to or implanted at a treatment site in order to release the active agent over an extended period of time.
13. A method for treating a disease/medical condition of a patient, the method comprising administering a hydrogel, organogel or xerogel comprising the biodegradable microparticles according to any one of the first to third lists of items above to the patient in order to release the active agent over an extended period of time.
14. The system for use or method of treatment according to any one of items 9 to 12, wherein the treatment site is selected from the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrascleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroidal, tenon, cornea, cancer tissue, organ, prostate, breast, joint, subdural, dental, subcutaneous, carpal tunnel, perivascular, surgically created space or injury, void space, and potential space.
15. The system for use or method of treatment according to any one of items 9 to 13, wherein the disease/medical condition to be treated is an eye disease, such as back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy; or glaucoma, ocular hypertension, hyphemia, presbyopia, cataract, retinal vein occlusion, inflammation.

### SIXTH LIST OF ITEMS

1. A method for controlling the release of an active agent from biodegradable microparticles for sustained drug-delivery according to the first to third list of items above, the method comprising one or a combination of the following measures:
   selecting/adjusting the L/G ratio of the polylactic-co-glycolic acid (PLGA) units to adjust the hydrophobicity of the polymer matrix forming the microparticles;
   selecting/adjusting the L/G ratio of the polylactic-co-glycolic acid (PLGA) units to provide a sustained release of the active agent from the microparticles;
   s selecting/adjusting the molar ratio of the amounts of the first to second crosslinkable precursors to adjust the hydrophobicity of the polymer matrix forming the microparticles;
   selecting/adjusting the molar ratio of the amounts of the first to second crosslinkable precursors to provide a sustained release of the active agent from the microparticles;
   adding a third crosslinkable precursor that is less hydrolysable than the first and second, optionally varying the molar ratios of the first, second and/or third precursors upon forming the biodegradable microparticles;
   dispersing an active agent that has high water solubility in particulate form into the organogel of a biodegradable microparticles;
   selecting/adjusting the amount and type of oil in the organogel microparticles;
   selecting/adjusting the amount and/or particle size of the biodegradable microparticles to be included in the in the hydrogel, organogel xerogel.

1-65. (canceled)

66. Biodegradable microparticles for sustained release drug-delivery, comprising an active agent and a covalently and three-dimensionally crosslinked biodegradable polymer, wherein at least one of the following conditions is fulfilled:
the crosslinked biodegradable polymer includes at least one of crosslinked polylactic acid (PLA), polyglycolic acid (PGA), polylactic-co-glycolic acid (PLGA), poly(vinylpyrrolidone), poly-p-dioxanone, poly(trimethylene carbonate), polycaprolactone, and/or polyvinyl alcohol, or copolymers of any of these;
the biodegradable microparticles include an organogel comprising the crosslinked biodegradable polymer and an oil.

67. The biodegradable microparticles of claim 66, wherein the microparticles are microspheres having a substantially spheroidal shape, and/or
wherein the active agent is dispersed, embedded or encapsulated in the polymer or organogel.

68. The biodegradable microparticles of claim 66, wherein the organogel or crosslinked polymer is formed by chemically crosslinking at least one multifunctional precursor to form the covalently and three dimensionally crosslinked polymer matrix, optionally in the presence of an oil.

69. The biodegradable microparticles of claim 68, wherein the at least one precursor has a functionality for chemical crosslinking of greater than 2, such as 3 to 10, or 3 to 9, or 4 to 8, or 4, preferably wherein the at least one precursor is a dendrimer or multi-arm precursor having a core and from 2 to 10 arms, or 3 to 10 arms, 4 to 8 arms, or 4 or 6 arms, each arm comprising a polymer unit and having a terminus.

70. The biodegradable microparticles of claim 68, wherein the polymer units are selected from polyethylene glycol (PEG), polypropyleneglycol (PPG), polyvinyl alcohol, poly (vinylpyrrolidone), polylactic acid (PLA), polyglycolic acid (PGA), polylactic-co-glycolic acid (PLGA), poly-p-dioxanone, poly(trimethylene carbonate), polycaprolactone, random or block copolymers or combinations or mixtures of any of these, or one or more units of polyaminoacids, glycosaminoglycans, polysaccharides, or proteins.

71. The biodegradable microparticles of claim 68, wherein the biodegradable, covalently and three-dimensionally crosslinked polymer matrix comprises a plurality of hydrophobic polymer units and/or hydrophilic polymer units.

72. The biodegradable microparticles of claim 71, wherein the hydrophobic polymer units are selected from at least one of polylactic acid (PLA), and polylactic-co-glycolic acid (PLGA) units, preferably polylactic acid (PLA); and the hydrophilic polymer units are selected from at least one of polyethylene glycol units, polypropylene glycol units, or polyglycolic acid (PGA), preferably polyethylene glycol units.

73. The biodegradable microparticles of claim 66, wherein the covalently and three-dimensionally crosslinked polymer comprises or consists of polylactic-co-glycolic acid (PLGA) units, or polylactic acid (PLA).

74. The biodegradable microparticles of claim 72, wherein the polylactic-co-glycolic acid (PLGA) units have an L/G ratio (in % L or G units) ranging from 0:100 to 100:0, or 1:99 to 99:1, or 10:90 to 90:10, or 25:75 to 75:25, preferably 50:50.

75. The biodegradable microparticles of claim 68, further including at least one crosslinker having at least two functional groups, or more than two functional groups, preferably a small molecule amine such as tris(2-aminoethyl)amine (TAEA) or trilysine.

76. The biodegradable microparticles of claim 68, wherein the organogel comprises at least two crosslinkable dendrimer or multi-arm precursors crosslinked with each other, preferably wherein the dendrimer or multi-arm precursor comprises a functional group on at least 3 of its arm termini, or on each terminus.

77. The biodegradable microparticles of claim 75, wherein the polymer matrix is formed from a first multi-arm precursor comprising a first functional group, and a second multi-arm precursor or crosslinker comprising a second functional group, the functional groups being located at the terminus of the arms of the precursor or crosslinker, wherein the first or second functional group may be directly grafted to the precursor terminus, or via a linker molecule.

78. The biodegradable microparticles of claim 77, wherein each of the first functional group and the second functional group is selected from an electrophile and a nucleophile, functional groups for click chemistry, functional groups for cycloadditions, particularly 1,3 dipolar cycloadditions, hetero-Diels-Alder cycloadditions, functional groups for nucleophilic ring openings, functional groups for non-aldol type carbonyl reactions, functional groups for addition reactions to carbon-carbon multiple bonds, polymerizable vinyl groups, or combinations thereof.

79. The biodegradable microparticles of claim 78, wherein each of the first functional group and the second functional group is selected from an electrophile and a nucleophile, and the reaction between the first functional group and second functional group is an electrophile-nucleophile reaction that forms a covalent bond.

80. The biodegradable microparticles of claim 66, wherein the polymer is covalently crosslinked by linkages between polymer units; and/or wherein the linkages are selected from the group consisting of amine, amide, urethane, ester, anhydride, ether, acetal, ketal, nitrile, isonitrile, isothiocyanate, isourea, hydrazone, oxime, or imine bonds, and combinations thereof.

81. The biodegradable microparticles of claim 66, wherein the active agent is selected from at least one of a therapeutically active agent or a diagnostically active agent, or combinations thereof.

82. The biodegradable microparticles of claim 66, wherein the therapeutically active agent is selected from steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; complement inhibitors such as avacincaptad pegol; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, viruses, viruses for gene delivery such as AAV, etc., or any combinations thereof.

83. The biodegradable microparticles of claim 66, wherein the microparticles have a particle size (diameter) of 0.1-1000 μm, or 1 to 150 μm, 1 to 100 μm, 20 to 75 μm, 10 to 106 μm or 20 to 55 μm, determined by sieving, or have an average diameter ranging from 0.1-1000 μm, or 1 to 150 μm, 1 to 100 μm, 20 to 75 μm, 10 to 106 μm or 20 to 55 μm, determined by laser diffraction; or wherein the microparticles have a particle size distribution such as a D50 particle size of less than about 100 μm, or less than about 50 μm, or less than about 20 μm and/or a D90 particle size of less than about 200 μm, or less than about 50 μm, or a D90 particle size of about 100 μm or less, or 30 μm or less and/or a D90 particle size of about 20 μm or less, determined by laser diffraction.

84. A method for manufacturing the biodegradable microparticles for sustained release drug-delivery according to any one of the preceding claims, the method being selected from one of the following techniques: emulsion solvent evaporation-extraction, emulsion solvent diffusion, supercritical fluid emulsion, coacervation, spray drying, hydrogel template, microfluidic systems, membrane extrusion emulsification, particle replication in non-wetting templates (PRINT) technique, electro hydrodynamic atomization (EHDA) or electro-spraying, or particles obtained from gas saturated solutions (PGSS) method or by 3D printing, the method comprising the steps of:
(1) forming a gel comprising a covalently crosslinked polymer in the presence of at least one active agent, optionally at least one oil and optionally a first solvent,
(2) producing microparticles wherein the at least one active agent is dispersed within the covalently crosslinked polymer, and
(3) optionally removing solvent.

85. A sustained release, biodegradable drug-delivery system, comprising the biodegradable microparticles for sustained release drug-delivery according to claim 66 wherein the biodegradable microparticles are incorporated into a hydrogel, xerogel or organogel, optionally by using extrusion methods or by 3D printing.

86. The sustained release, biodegradable drug-delivery system of claim 85, for coating a medical implant or for use as a medical implant.

87. A method for treating a disease/medical condition of a patient, the method comprising administering a hydrogel, organogel or xerogel comprising the biodegradable microparticles according to claim 66, to the patient in order to release the active agent over an extended period of time.

88. The method of treatment according to claim 23, wherein the treatment site is selected from the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrascleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroidal, tenon, cornea, cancer tissue, organ, prostate, breast, joint, subdural, dental, subcutaneous, carpal tunnel, perivascular, surgically created space or injury, void space, and potential space.

89. The method of treatment according to claim 23, wherein the disease/medical condition to be treated is an eye disease, such as back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy; or glaucoma, ocular hypertension, hyphemia, presbyopia, cataract, retinal vein occlusion, inflammation.
